(19) United States
(12) Patent Application Publication
(10) Pub. No.: US 2011/0300068 A1
(43) Pub. Date: Dec. 8, 2011

(54) AGENT FOR PROPHYLAXIS OR TREATMENT OF CANCER

(75) Inventors: Tatsuya Watanabe, Kanagawa (JP); Shinichi Kondo, Kanagawa (JP); Ryosuke Katsuyama, Kanagawa (JP)

(73) Assignee: Takeda Pharmaceutical Company Limited, Osaka (JP)

(21) Appl. No.: 13/138,146

(22) PCT Filed: Jan. 12, 2010
(86) PCT No.: PCT/JP2010/050460

§ 371 Date: Aug. 24, 2011

Related U.S. Application Data
(60) Provisional application No. 61/204,827, filed on Jan. 12, 2009.

(51) Int. Cl. A61K 51/10; A61P 35/00; G01N 33/574; A61K 49/00; C07K 16/22; A61K 39/395

(52) U.S. Cl. 424/1.49; 530/389.2; 530/388.24; 530/387.3; 424/158.1; 436/501; 424/9.1; 435/7.92

(57) ABSTRACT

The present invention provides an agent for the prophylaxis or treatment of kidney cancer or urinary bladder cancer, and a diagnostic reagent for these cancers which comprise an antibody against nerve growth factor 2/neurotrophin-3 or a partial peptide thereof or a salt thereof, which antibody neutralizes nerve growth factor 2/neurotrophin-3 and does not cross-react with the Nerve Growth Factor.

### TECHNICAL FIELD

The present invention relates to a novel use of a neutralizing antibody against nerve growth factor 2/neurotrophin-3, more particularly, use for the prophylaxis or treatment as well as diagnosis of particular cancers (urinary bladder cancer, kidney cancer and the like).

### BACKGROUND OF THE INVENTION

Nerve growth factor 2 (NGF2; also known as neurotrophin-3 (NT-3); hereinafter abbreviated as NGF2/NT-3), a member of the neurotrophic factor (neurotrophin) family, is closely associated with both Nerve Growth Factor (NGF; also abbreviated as NGF1) and brain derived neurotrophic factor (BDNF). It has been described that NGF2/NT-3 protein can be used for the treatment of nerve damage and other neuropathies because it promotes the survival, proliferation and differentiation of mammalian nerve cells and induces various proteins, enzymes and the like (patent documents 1-3). Meanwhile, it has been suggested that excess action of NGF2/NT-3 may induce abnormal regeneration of sensor neurons, chronic pain, or neuropathies such as dementia, and it has been described that an anti-NGF2/NT-3 neutralizing antibody can be used to treat these diseases (patent documents 4-6).

It has been reported that the expression of NGF2/NT-3 is elevated in some particular cancers, and that an anti-NGF2/NT-3 neutralizing antibody is capable of suppressing the growth of these cancers. For example, Sheila J. Miknyoczki et al. described that a rabbit polyclonal neutralizing antibody against recombinant NGF2/NT-3 suppressed the proliferation of prostate cancer cells in xenograft experiments (patent document 7 and non-patent document 1).

However, regarding the association of NGF2/NT-3 with kidney cancer and urinary bladder cancer, and the use of an anti-NGF2/NT-3 neutralizing antibody in the treatment and diagnosis of these cancers, nothing has been known so far.
patent document 1: U.S. Pat. No. 5,656,435
patent document 2: U.S. Pat. No. 5,712,100
patent document 3: EP 0418590
patent document 4: U.S. Pat. No. 5,180,820
patent document 5: U.S. Pat. No. 6,933,276
patent document 6: JP-A-6-189787
patent document 7: U.S. Pat. No. 6,548,062
non-patent document 1: Clinical Cancer Research 8: 1924-1931, 2002

### DISCLOSURE OF THE INVENTION

### Problems to be Solved by the Invention

An object of the present invention is to provide a novel agent for the prophylaxis or treatment of kidney cancer and urinary bladder cancer, and a diagnostic reagent therefor. Another object of the present invention is to provide a novel pharmaceutical use of an anti-NGF2/NT-3 neutralizing antibody.

### Means of Solving the Problems

The present inventors have conducted intensive studies in an attempt to solve the above-mentioned problems and found that the anti-human NGF2/NT-3 neutralizing antibody (3W3 antibody) described in JP-A-6-189787 suppresses the growth of kidney cancer and urinary bladder cancer cells.

Further investigation was carried out based on these findings, thereby the inventors completed the invention.

Accordingly, the present invention relates to the following.

[1] An agent for the prophylaxis or treatment of kidney cancer, comprising an antibody against NGF2/NT-3 or a partial peptide thereof or a salt thereof, which antibody neutralizes nerve growth factor 2/neurotrophin-3 (also abbreviated as NGF2/NT-3) and does not cross-react with the NGF.
[2] An agent for the prophylaxis or treatment of urinary bladder cancer, comprising an antibody against NGF2/NT-3 or a partial peptide thereof or a salt thereof, which antibody neutralizes NGF2/NT-3 and does not cross-react with the NGF.
[3] The agent of [1] or [2], wherein the antibody is a monoclonal antibody producible by the hybridoma 3W3 cell line (FERM BP-3932).
[4] The agent of [1] or [2], wherein the NGF2/NT-3 is human NGF2/NT-3.
[5] The agent of [1] or [2], wherein the antibody is a humanized antibody or a human antibody.
[6] The agent of [1] or [2], wherein the antibody is a human/non-human chimeric antibody.
[7] A diagnostic reagent of kidney cancer, comprising an antibody against NGF2/NT-3 or a partial peptide thereof or a salt thereof, which antibody neutralizes NGF2/NT-3 and does not cross-react with the NGF.
[8] A diagnostic reagent of urinary bladder cancer, comprising an antibody against NGF2/NT-3 or a partial peptide thereof or a salt thereof, which antibody neutralizes NGF2/NT-3 and does not cross-react with the NGF.
[9] A method for the prophylaxis or treatment of kidney cancer, comprising administering to a mammal an effective amount of an antibody against NGF2/NT-3 or a partial peptide thereof or a salt thereof, which antibody neutralizes NGF2/NT-3 and does not cross-react with the NGF.
[10] A method for the prophylaxis or treatment of urinary bladder cancer, comprising administering to a mammal an effective amount of an antibody against NGF2/NT-3 or a partial peptide thereof or a salt thereof, which antibody neutralizes NGF2/NT-3 and does not cross-react with the NGF.
[11] A method of diagnosing kidney cancer, comprising using an antibody against NGF2/NT-3 or a partial peptide thereof or a salt thereof, which antibody neutralizes NGF2/NT-3 and does not cross-react with the NGF.
[12] A method of diagnosing urinary bladder cancer, comprising using an antibody against NGF2/NT-3 or a partial peptide thereof or a salt thereof, which antibody neutralizes NGF2/NT-3 and does not cross-react with the NGF.
[13] Use of an antibody against NGF2/NT-3 or a partial peptide thereof or a salt thereof, which antibody neutralizes NGF2/NT-3 and does not cross-react with the NGF, for the production of an agent for the prophylaxis or treatment of kidney cancer.
[14] Use of an antibody against NGF2/NT-3 or a partial peptide thereof or a salt thereof, which antibody neutralizes NGF2/NT-3 and does not cross-react with the NGF, for the production of an agent for the prophylaxis or treatment of urinary bladder cancer.
[15] Use of an antibody against NGF2/NT-3 or a partial peptide thereof or a salt thereof, which antibody neutralizes NGF2/NT-3 and does not cross-react with the NGF, for the production of a diagnostic reagent of kidney cancer.
[16] Use of an antibody against NGF2/NT-3 or a partial peptide thereof or a salt thereof, which antibody neutralizes NGF2/NT-3 and does not cross-react with the NGF, for the production of a diagnostic reagent of urinary bladder cancer.

### Effect of the Invention

Since an anti-NGF2/NT-3 neutralizing antibody can suppress growth of kidney cancer and urinary bladder cancer cells, a pharmaceutical agent comprising the antibody is useful as an agent for the prophylaxis and/or treatment of these cancers. In addition, an anti-NGF2/NT-3 antibody can also be used for the diagnosis of kidney cancer and urinary bladder cancer, screening for a pharmaceutical agent that exhibits a prophylactic and/or treatment effect on these cancers by suppressing the expression of NGF2/NT-3, and the like.

### BRIEF DESCRIPTION OF THE DRAWINGS

FIG. 1 shows a NGF2/NT-3 neutralizing activity of the 3W3 antibody.

FIG. 2 shows the amino acid sequence and predicted CDR of the 3W3 antibody heavy chain.

FIG. 3 shows the amino acid sequence and predicted CDR of the 3W3 antibody light chain.

FIG. 4 (i.e., 4A and 4B) shows an antitumor activity of the 3W3 antibody on urinary bladder cancer cell. FIG. 4A shows tumor size changes after administration of control antibody (-♦-) or 3W3 antibody (-▪-), and FIG. 4B shows body weight changes after administration of each antibody.

FIG. 5 (i.e., 5A and 5B) shows an antitumor activity of the 3W3 antibody on kidney cancer cell. FIG. 5A shows tumor size changes after administration of control antibody (-♦-) or 3W3 antibody (-▪-), and FIG. 5B shows body weight changes after administration of each antibody.

FIG. 6 shows an antitumor activity of the 3W3 antibody on small-cell lung cancer cell. FIG. 6A shows tumor size changes after administration of control antibody or 3W3 antibody (1 mg or 10 mg) administration group. FIG. 6B shows body weight changes after administration of each antibody.

### DETAILED DESCRIPTION OF THE INVENTION

### Anti-NGF2/NT-3 Antibody

The antibody used in the present invention is an antibody against NGF2/NT-3 or a partial peptide thereof or a salt thereof, which antibody neutralizes NGF2/NT-3 and does not cross-react with other neurotrophic factors such as the Nerve Growth Factor (i.e, NGF1) (specifically recognizes NGF2/NT-3), hereinafter also referred to as the antibody of the present invention.

NGF2/NT-3, which is recognized by the antibody of the present invention, is a protein comprising the same or substantially the same amino acid sequence as the amino acid sequence shown by amino acid numbers 123 to 241 in the amino acid sequence shown by SEQ ID NO:2. Here, the amino acid sequence shown by SEQ ID NO:2 indicates the amino acid sequence of the initial translation product (preproprotein) of human NGF2/NT-3 isoform 1 (Refseq number: NP_{—}001096124.1), in which the amino acid sequence shown by amino acid numbers 123 to 241 indicates the amino acid sequence of mature human NGF2/NT-3. Hereinafter, unless otherwise stated, the amino acid sequence of mature human NGF2/NT-3 is simply abbreviated as “the amino acid sequence shown by SEQ ID NO:2”. “Substantially the same amino acid sequence as the amino acid sequence shown by SEQ ID NO:2” means the amino acid sequence of a naturally occurring protein that is not completely identical to the amino acid sequence shown by SEQ ID NO:2, but possesses the same function, like other isoforms of human NGF2/NT-3 [however, currently known human NGF2/NT-3 isoform 2 (Refseq number: NP_{—}002518.1) is completely identical to isoform 1 except that 13 amino acid residues on the N-terminus side of the signal peptide of isoform 1 are lacked, so these are indistinguishable as mature proteins], allele mutants (e.g., those of allele frequency of less than 1%), gene polymorphs (e.g., those of minor allele frequency of 1% or more) and the like.

The above-described mutated or polymorphic protein comprises an amino acid sequence having a homology of normally about 90% or more, preferably about 95% or more, more preferably about 97% or more, still more preferably about 98% or more, most preferably about 99% or more, to the base sequence shown by SEQ ID NO:2. Here, the “homology” means a ratio (%) of the same amino acid and similar amino acid residue to the total overlapped amino acid residue, in the best alignment when two amino acid sequences are aligned with the use of a mathematical algorithm commonly known in the technical field (preferably, the algorithm is obtained by consider allowing gaps on one or both side of the sequence for the best alignment). The term “similar amino acid” refers to an amino acid similar in its physiochemical properties, and examples include amino acids classified in a same group such as aromatic amino acid (Phe, Trp, Tyr), aliphatic amino acid (Gly, Ala, Leu, Ile, Val), polar amino acid (Gln, Asn), basic amino acid (Lys, Arg, His), acidic amino acid (Glu, Asp), amino acid including a hydroxyl group (Ser, Thr), amino acid having a short side chain (Ala, Ser, Thr, Met), and the like. A substitution by such similar amino acid is expected to give no change in the phenotype of protein (thus is a conservative amino acid substitution). A specific example of the conservative amino acid substitution is well-known in the technical field, and is disclosed in various documents (e.g., refer Bowie et al., Science, 247: 1306-1310 (1990)).

Homology of the amino acid sequences in the present description can be measured under the following conditions (an expectation value=10; gaps are allowed; matrix=BLOSUM62; filtering=OFF) using a homology scoring algorithm NCBI BLAST (National Center for Biotechnology Information Basic Local Alignment Search Tool). Other algorithm for determining homology of the amino acid sequence is exemplified by an algorithm disclosed in Karlin et al., Proc. Natl. Acad. Sci. USA, 90: 5873-5877 (1993) (this algorithm is incorporated in NBLAST and XBLAST program (version 2.0) (Altschul et al., Nucleic Acids Res., 25: 3389-3402 (1997))]; an algorithm disclosed in Needleman et al., J. Mol. Biol., 48: 444-453 (1970) [This algorithm is incorporated in a GAP program in a GCG software package]; an algorithm disclosed in Myers and Miller, CABIOS, 4: 11-17 (1988) [This algorithm is incorporated in ALIGN program (version 2.0) which is a part of a CGC sequence alignment software package]; an algorithm disclosed in Pearson et al., Proc. Natl. Acad. Sci. USA, 85: 2444-2448 (1988) [This algorithm is incorporated in an FASTA program in a GCG software package], etc., and these may be also preferably used.

The above-described mutated or polymorphic protein normally comprises (i) an amino acid sequence resulting from deletion of about 1 to 10, preferably one to several (5, 4, 3 or 2), amino acids in the amino acid sequence shown by SEQ ID NO:2, (ii) an amino acid sequence resulting from addition of about 1 to 10, preferably one to several (5, 4, 3 or 2), amino acids in the amino acid sequence shown by SEQ ID NO:2, (iii) an amino acid sequence resulting from insertion of about 1 to 10, preferably one to several (5, 4, 3 or 2), amino acids in the amino acid sequence shown by SEQ ID NO:2, (iv) an amino acid sequence resulting from substitution of about 1 to 10, preferably one to several (5, 4, 3 or 2), amino acids in the amino acid sequence shown by SEQ ID NO:2 by other amino acids, or (v) an amino acid sequence being a combination thereof.

Where the amino acid sequence is inserted, deleted or substituted as described above, the position of its insertion, deletion, or substitution is not particularly limited.

The antibody of the present invention is also widely applicable to non-human warm-blooded animals (e.g., monkeys, bovines, horses, pigs, sheep, goats, dogs, cats, rabbits, guinea pigs, hamsters, rats, mice, fowls and the like). It is evident, therefore, that the NGF2/NT-3 entities recognized by the antibody of the present invention include not only the above-described human protein, but also an orthologue thereof in another warm-blooded animal. In this case, the homology between the orthologue and the human gene is not subject to limitations, and is desirably as high as possible; for example, the homology may be about 70% or more, preferably about 80%, more preferably about 90% or more. For an orthologue in another mammal, sequence information can be acquired by searching a non-human mammal protein database using BLAST or FASTA, with the amino acid sequence shown by SEQ ID NO:2 per se or a public database accession number (e.g., in the case of Refseq No.: NP_{—}001096124.1 and the like) as the query, or by performing a search using, for example, the Mouse Genome Informatics (http://www.informatics.jax.org/) provided by the Jackson Laboratories, with an accession number or gene symbol/gene name as the keyword, and accessing the Mammalian Orthology information on the hit data, or the like. As far as known at present, the amino acid sequence of mature NGF2/NT-3 is completely the same among all mammals (e.g., humans, chimpanzees, bovines, dogs, rats, mice and the like).

In the present description, the proteins and peptides are represented in accordance with the conventional way of describing peptides, that is, the N-terminus (amino terminus) at the left hand and the C-terminus (carboxyl terminus) at the right hand.

NGF2/NT-3 containing an amino acid sequence shown by SEQ ID NO:2 may have the C-terminus in any form of a carboxyl group (—COOH), a carboxylate (—COO⁻), an amide (—CONH₂) and an ester (—COOR). Herein, examples of the ester group shown by R include a C₁₋₆ alkyl group such as methyl, ethyl, n-propyl, isopropyl, n-butyl, etc.; a C₃₋₈ cycloalkyl group such as cyclopentyl, cyclohexyl, etc.; a C₆₋₁₂ aryl group such as phenyl, α-naphthyl, etc.; a C₇₋₁₄ aralkyl such as a phenyl-C₁₋₂ alkyl group, e.g., benzyl, phenethyl, etc.; an α-naphthyl-C₁₋₂ alkyl group such as α-naphthylmethyl, etc.; pivaloyloxymethyl and the like.

Where the NGF2/NT-3 contains a carboxyl group (or a carboxylate) at a position other than the C-terminus, the carboxyl group may be amidated or esterified and such an amide or ester is also included within the NGF2/NT-3 in the present invention. Examples of the ester group in this case may be the C-terminal esters described above, etc.

Furthermore, examples of the NGF2/NT-3 include variants wherein the amino group at the N-terminal amino acid residues (e.g., methionine residue) is protected with a protecting group (e.g., a C₁₋₆ acyl group such as a C₁₋₆ alkanoyl group, e.g., formyl group, acetyl group, etc.); those wherein the N-terminal region is cleaved in vivo and the glutamyl group thus formed is pyroglutaminated; those wherein a substituent (e.g., —OH, —SH, amino group, imidazole group, indole group, guanidino group, etc.) on the side chain of an amino acid in the molecule is protected with a suitable protecting group (e.g., a C₁₋₆ acyl group such as a C₁₋₆ alkanoyl group, e.g., formyl group, acetyl group, etc.), or conjugated proteins such as so-called glycoproteins having sugar chains; etc.

The partial peptide of NGF2/NT-3 recognized by the antibody of the present invention may be any peptide having a partial amino acid sequence of the aforementioned NGF2/NT-3 protein, and possessing antigenicity; examples include a peptide comprising one or two kinds or more of three or more, preferably six or more, continuous amino acids of the same or substantially the same amino acid sequence shown by amino acid numbers 123 to 241 in the amino acid sequence shown by SEQ ID NO:2 and the like.

The partial peptide, like NGF2/NT-3 protein, may have the C-terminus in any form of a carboxyl group (—COOH), a carboxylate (—COO⁻), an amide (—CONH₂) or an ester (—COOR). Herein, as the “R” in ester, same ones as in the NGF2/NT-3 can be exemplified. Where the peptide contains a carboxyl group (or a carboxylate) at a position other than the C-terminus, the carboxyl group may be amidated or esterified and such an amide or ester is also included within the partial peptide of NGF2/NT-3. Examples of the ester group in this case may be the C-terminal esters described above, etc.

Furthermore, the partial peptide used in the present invention includes those wherein the amino group at the N-terminal amino acid residues (e.g., methionine residue) is protected with a protecting group; those wherein the N-terminal region is cleaved in vivo and the glutamyl group thus formed is pyroglutaminated; those wherein a substituent on the side chain of an amino acid in the molecule is protected with a suitable protecting group, or conjugated peptides such as so-called glycopeptides having sugar chains; etc., as in the NGF2/NT-3 protein described above.

The partial peptide, like the NGF2/NT-3 protein, can be used as an antigen for producing the antibody of the present invention.

The NGF2/NT-3 or its partial peptide may be a free form, or a salt form (it is same throughout the present description unless otherwise specified). As the salts, salts with physiologically acceptable acids (e.g., inorganic acids, organic acids, etc.) or bases (e.g., alkali metals, alkaline earth metal etc.), preferably physiologically acceptable acid addition salts can be used. Examples of such salts include salts with inorganic acids (e.g., hydrochloric acid, phosphoric acid, hydrobromic acid, sulfuric acid), salts with organic acids (e.g., acetic acid, formic acid, propionic acid, fumaric acid, maleic acid, succinic acid, tartaric acid, citric acid, malic acid, oxalic acid, benzoic acid, methanesulfonic acid, benzenesulfonic acid) and the like.

The antibody of the present invention may be a polyclonal antibody or a monoclonal antibody, as far as it is capable of recognizing the above-described NGF2/NT-3 or partial peptide thereof. The isotype of the antibody is not subject to limitations, and is preferably IgG, IgM or IgA, more preferably IgG.

The antibody of the present invention is not subject to limitation, as long as it has at least a complementality determining region (CDR) for specifically recognizing and binding to the target antigen; in addition to the whole antibody molecule, the antibody may, for example, be a fragment such as Fab, Fab′, or F(ab′)₂, a genetically engineered conjugate molecule such as scFv, scFv-Fc, minibody, or diabody, or a derivative thereof modified with a molecule having protein stabilizing action, such as polyethylene glycol (PEG), or the like, and the like.

The antibody of the present invention can be produced by a method of antibody or antiserum production known per se. Typical examples of the method of preparing an immunogen for the antibody of the present invention and a method of producing the antibody are described below.

### (1) Preparation of Antigen

The antigen used to prepare the antibody of the present invention may be any one of the above-described NGF2/NT-3 or partial peptide thereof, a (synthetic) peptide having one or two kinds or more of the same antigen determinant as that thereof and the like (hereinafter these are sometimes simply referred to as the antigen of the present invention).

NGF2/NT-3 or a partial peptide thereof is produced by, for example, (a) preparing the same from a tissue or cells of a warm-blooded animal such as a human, a monkey, a rat, a mouse, or a fowl using a publicly known method or a method based thereon, (b) chemically synthesizing the same by a publicly known method of peptide synthesis using a peptide synthesizer and the like, (c) culturing a transformant comprising a DNA that encodes NGF2/NT-3 or a partial peptide thereof, or (d) biochemically synthesizing the same with a nucleic acid that encodes NGF2/NT-3 or a partial peptide thereof as the template using a cell-free transcription/translation system.

(a) Preparation of NGF2/NT-3 from Warm-Blooded Animal tissue or cells

NGF2/NT-3 can be produced from cells [for example, hepatocytes, splenocytes, nerve cells, glial cells, β cells of pancreas, bone marrow cells, mesangial cells, Langerhans' cells, epidermic cells, epithelial cells, goblet cells, endothelial cells, smooth muscle cells, fibroblasts, fibrocytes, myocytes, adipocytes, immune cells (e.g., macrophages, T cells, B cells, natural killer cells, natural killer T cells, mast cells, neutrophils, basophils, eosinophils, monocytes), megakaryocytes, synovial cells, chondrocytes, bone cells, osteoblasts, osteoclasts, mammary cells, or interstitial cells; or the corresponding progenitor cells, stem cells, cancer cells and the like] or any tissues where such cells are present [for example, brain or each part of brain (e.g., olfactory bulb, amygdaloid nucleus, basal ganglia, hippocampus, thalamus, hypothalamus, cerebral cortex, medulla oblongata, cerebellum), spinal cord, hypophysis, stomach, pancreas, kidney, liver, gonad, thyroid, gall-bladder, bone marrow, adrenal gland, skin, muscles (e.g., smooth muscle, skeletal muscle), lung, gastrointestinal tract (e.g., large intestine, small intestine), blood vessel, heart, thymus, spleen, submandibular gland, peripheral blood, prostate, testis, ovary, placenta, uterus, bone, joint, adipose tissue (e.g., white adipose tissue, brown adipose tissue) and the like] of humans or other warm-blooded animals by a method of protein purification known per se. For example, a tissue or cells of a warm-blooded animal are cultured in a medium, and the resulting supernatant fraction thereof can be used as the antigen as it is, or after being concentrated as required. Alternatively, the NGF2/NT-3 obtained can also be isolated and purified from the supernatant by a combination of salting out, dialysis, chromatographies such as gel filtration chromatography, reversed phase chromatography, ion-exchange chromatography, and affinity chromatography, and the like. The NGF2/NT-3 obtained can be used as the immunogen as is, and can also be used as the immunogen in the form of a partial peptide prepared by limited degradation using a peptidase and the like.

When the thus-obtained NGF2/NT-3 or partial peptide thereof is a free form, it can be converted to an appropriate salt by a publicly known method or a method based thereon; conversely, when the protein or the partial peptide is obtained as a salt, it can be converted to the free form or another salt by a publicly known method or a method based thereon.

### (b) Chemical Preparation of the Antigen of the Present Invention

Examples of the synthetic peptide include peptides having the same structure as NGF2/NT-3, purified from a natural material using the method (a) above; specifically, a peptide comprising one or two kinds or more of amino acid sequences at an optionally chosen site, consisting of three or more, preferably six or more, continuous amino acids, in the amino acid sequence of the protein, and the like are used.

For the methods for peptide synthesis, for example, either solid phase synthesis or liquid phase synthesis may be used. That is, the partial peptide or amino acids that can construct the NGF2/NT-3 or partial peptide are condensed with the remaining part. Where the product contains protecting groups, these protecting groups are removed to give the desired peptide. Publicly known methods for condensation and elimination of the protecting groups are described in (i) to (v) below.

### (i) M. Bodanszky & M. A. Ondetti: Peptide Synthesis, Interscience Publishers, New York (1966)

### (ii) Schroeder & Luebke: The Peptide, Academic Press, New York (1965)

(iii) Nobuo Izumiya, et al.: Peptide Gosei-no-Kiso to Jikken (Basics and experiments of peptide synthesis), published by Maruzen Co. (1975)

### (iv) Haruaki Yajima & Shunpei Sakakibara: Seikagaku Jikken Koza (Biochemical Experiment) 1, Tanpakushitsu no Kagaku (Chemistry of Proteins) IV, 205 (1977)

(v) Haruaki Yajima ed.: Zoku Iyakuhin no Kaihatsu (A sequel to Development of Pharmaceuticals), Vol. 14, Peptide Synthesis, published by Hirokawa Shoten

The peptide obtained in the above manner may be purified and isolated by a combination of conventional purification methods such as solvent extraction, distillation, column chromatography, liquid chromatography and recrystallization to give the partial peptide used in the present invention.

When the partial peptide obtained by the above methods is in a free form, the partial peptide can be converted into an appropriate salt by a publicly known method or its modification; conversely when the partial peptide is obtained in a salt form, it can be converted into a free form or other different salt form by a publicly known method or its modification.

### (c) Where the Antigen of the Present Invention is Produced Using the DNA-Bearing Transformants

DNA encoding NGF2/NT-3 or a partial peptide thereof can be produced in accordance with publicly known cloning techniques [e.g., the method described in Molecular Cloning (2nd ed., J. Sambrook et al., Cold Spring Harbor Lab. Press, 1989), etc.]. The cloning method refers to (1) a method in which a DNA encoding NGF2/NT-3 or a partial peptide thereof is isolated from the cDNA library according to a hybridization method and using a DNA probe designed based on a gene sequence (e.g., base sequence shown by SEQ ID NO:1) encoding NGF2/NT-3, and (2) a method in which DNAs encoding the NGF2/NT-3 or a partial peptide thereof are produced from cDNA as a template by PCR using DNA primers designed based on the gene sequence encoding the NGF2/NT-3, and the DNA are inserted into an appropriate expression vector as a host. Incubating the transformants which is obtained from host by being transformed with the expression vector, in appropriate medium, the desired antigen can be obtained.

(d) When a cell-free transcription/translation system is utilized, a method for synthesizing an mRNA by using an expression vector incorporating a DNA that encodes the antigen or a fragment thereof (e.g., an expression vector wherein the DNA is placed under the control of the T7 or SP6 promoter and the like, and the like) as the template, that was prepared by the same method as (c) above, a transcription reaction mixture comprising an RNA polymerase matching the promoter, and its substrates (NTPs); and thereafter performing a translation reaction with the mRNA as the template using a known cell-free translation system (e.g., E. coli, rabbit reticulocytes, extract from wheat germ etc.), and the like can be mentioned. By adjusting the salt concentration and the like appropriately, the transcription reaction and the translation reaction can also be carried out in the same reaction mixture at one time.

As the immunogen, a whole mature NGF2/NT-3 or a partial peptide can be used. As examples of the partial peptide, those comprising 3 or more continuous amino acid residues, preferably those comprising 4 or more, more preferably 5 or more, still more preferably 6 or more continuous amino acid residues, can be mentioned. Alternatively, as examples of the partial peptide, those comprising 20 or less continuous amino acid residues, preferably those comprising 18 or less, more preferably 15 or less, still more preferably 12 or less continuous amino acid residues, can be mentioned. A portion of these amino acid residues (e.g., 1 to several residues) may be substituted with a substituent group (e.g., Cys, hydroxyl group, etc.). The peptide used as the immunogen has an amino acid sequence comprising one to several such partial amino acid sequences.

Warm-blooded animal cells itself which express the NGF2/NT-3 or a partial peptide thereof can also be used directly as the antigen. As the warm-blooded animal cells, there can be used the naturally occurring cells as described in (a) above, cells transformed by the methods as described in (c) above, etc. Hosts used for the transformation may be any cells as long as they are the cells collected from human, monkey, rat, mouse, hamster, fowl etc. and preferably used are HEK293, COS7, CHO-K1, NIH3T3, Balb3T3, FM3A, L929, SP2/0, P3U1, B16, P388, or the like. Naturally occurring warm-blooded animal cells or transformed warm-blooded animal cells, which express the NGF2/NT-3 or a partial peptide thereof, can be injected to an animal to be immunized as a suspension of the cells in a medium used for tissue culture (e.g., RPMI 1640) or buffer (e.g., Hanks' Balanced Salt Solution). Immunization may be done by any method, as long as it can stimulate antibody production, and preferably used are intravenous injection, intraperitoneal injection, intramuscular injection, subcutaneous injection, etc.

In still another embodiment, it is possible to use a DNA immunization method wherein an expression vector incorporating a DNA that encodes NGF2/NT-3 or a partial peptide thereof is introduced to an animal to be immunized by direct transduction to produce NGF2/NT-3 or a partial peptide thereof in the body of the animal. This method can be used preferably in, for example, preparing an antibody that recognizes the higher structure of NGF2/NT-3.

The antigen of the present invention permit direct use for immunization in an insolubilized form, as long as it has immunogenicity; when an antigen of low molecular weight (e.g., molecular weight about 3,000 or less) having only one to several antigenic determinants in the molecule thereof is used, it can be used for immunization in the form of a complex bound or adsorbed to a suitable carrier because these antigens are normally hapten molecules of low immunogenicity. As the carrier, a naturally occurring or synthetic polymer can be used. As examples of the naturally occurring polymer, serum albumin of a mammal such as bovine, rabbit, or human, thyroglobulin of a mammal such as bovine or rabbit, ovalbumin of, for example, fowl, hemoglobin of a mammal such as bovine, rabbit, human, or sheep, keyhole limpet hemocyanin (KLH) and the like can be used. As examples of the synthetic polymer, various latexes of polymers or copolymers of polyamino acids, polystyrenes, polyacryls, polyvinyls, polypropylenes and the like, and the like can be mentioned.

A mixing ratio of the carrier to the hapten may be in any ratio of any type, as long as the antibody to the antigen of the present invention can be efficiently produced. A high molecular carrier conventionally used to produce an antibody to a hapten may be used in a weight ratio of 0.1 to 100 based on 1 of hapten.

For coupling of the hapten and the carrier, a variety of condensing agents can be used. Examples of the condensing agents, which are advantageously employed, are diazonium compounds such as bis-diazotized benzidine etc. capable of crosslinking tyrosines, histidines or tryptophans; dialdehyde compounds such as glutaraldehyde, etc. capable of crosslinking amino groups with each other; diisocyanate compounds such as toluene-2,4-diisocyanate, etc.; dimaleimide compounds such as N,N′-o-phenylenedimaleimide, etc. capable of crosslinking thiols with each other; maleimide activated ester compounds capable of crosslinking an amino group with a thiol group; carbodiimide compounds capable of crosslinking an amino group with a carboxyl group; etc. In the crosslinking of amino groups with each other, one amino group is reacted with an activated ester reagent (e.g., N-succinimidyl 3-(2-pyridyldithio)propionate (SPDP), etc.) having dithiopyridyl group and then reduced to introduce the thiol group, whereas another amino group is introduced with a maleimide group using a maleimide activated ester reagent, and the two groups may be reacted with each other.

### (2) Preparation of Monoclonal Antibody

### (a) Preparation of Monoclonal Antibody Producing-Cell

An antigen is administered as is, or along with a carrier or a diluent, to a warm-blooded animal at a site enabling antibody production by the methods such as intraperitoneal injection, intravenous injection, subcutaneous injection, intradermal injection and the like. In order to increase antibody productivity upon the administration, Freund's complete adjuvant or Freund's incomplete adjuvant may be administered. Dosing is normally performed about two to 10 times in total every 1 to 6 weeks. As examples of the warm-blooded animal used, monkeys, rabbits, dogs, guinea pigs, mice, rats, hamsters, sheep, goats, donkeys and fowls can be mentioned. Although it is preferable to use a mammal of the same species as the recipient in order to avoid the problem of anti-Ig antibody production, mice and rats are generally preferably used for generating a monoclonal antibody.

Since artificial immunization to humans is ethically difficult, it is preferable, when the antibody of the present invention targets a human, (i) to obtain a human antibody by immunizing a human antibody-producing animal (e.g., mouse) produced according to a method described below, (ii) to produce a chimeric antibody, humanized antibody or fully human antibody according to a method described below, or (iii) to obtain a human antibody using in combination the in vitro immunization method and cell immortalization with virus, human-human (or human-mouse) hybridoma production technique, phage display method and the like. Note that the in vitro immunization method can also be used preferably as a method for obtaining an antibody against an antigen that is unstable and difficult to prepare in large amounts for the purpose of preparing a non-human animal-derived antibody, because there is the possibility of obtaining an antibody against an antigen for which antibody production is suppressed by ordinary immunization, because it is possible to obtain an antibody with an amount of antigen on the nanogram to microgram order, because immunization completes in several days, and for other reasons.

As the animal cells used in the in vitro immunization method, lymphocytes, preferably B-lymphocytes and the like, isolated from peripheral blood, spleen, lymph node and the like of a human and the above-described warm-blooded animals (preferably mouse or rat) can be mentioned. For example, in the case of mouse or rat cells, the spleen is extirpated from an about 4- to 12-week-old animal, and splenocytes are separated and rinsed with a appropriate medium [e.g., Dulbecco's modified Eagle medium (DMEM), RPMI1640 medium, Ham's F12 medium and the like], after which the splenocytes are suspended in an antigen-comprising medium supplemented with fetal bovine serum (FCS; about 5 to 20%) and cultured using a CO₂ incubator and the like for about 4 to 10 days. Examples of the antigen concentration include, but are not limited to, 0.05 to 5 pg. It is preferable to prepare a culture supernatant of thymocytes of an animal of the same strain (preferably at about 1 to 2 weeks of age) according to a conventional method, and to add the supernatant to the medium.

Since it is difficult to obtain a thymocyte culture supernatant in in vitro immunization of human cells, it is preferable to perform immunization by adding, to the medium, several kinds of cytokines such as IL-2, IL-4, IL-5, IL-6 and the like, and if necessary, an adjuvant substance (e.g., muramyldipeptide and the like) along with the antigen.

In preparing a monoclonal antibody, it is possible to establish an antibody-producing hybridoma by selecting an individual or cell population showing an elevated antibody titer from among antigen-immunized warm-blooded animals (e.g., mice, rats) or animal cells (e.g., human, mouse, rat), respectively; collecting spleens or lymph nodes at 2 to 5 days after the final immunization or collecting the cells after 4 to 10 days of cultivation after in vitro immunization to isolate antibody-producing cells; and fusing the isolated cells with myeloma cells. A measurement of serum antibody titer can be performed by, for example, reacting a labeled antigen and an antiserum, and thereafter determining the activity of the label bound to the antibody.

Although the myeloma cells are not subject to limitation, as long as they are capable of producing a hybridoma that secretes a large amount of antibody, those that do not produce or secrete the antibody per se are preferable, with greater preference given to those of high cell fusion efficiency. To facilitate hybridoma selection, it is preferable to use a cell line that is susceptible to HAT (hypoxanthine, aminopterin, thymidine). As examples of the mouse myeloma cells, NS-1, P3U1, SP2/0, AP-1 and the like can be mentioned; as examples of the rat myeloma cells, R210.RCY3, Y3-Ag 1.2.3 and the like can be mentioned; as examples of the human myeloma cells, SKO-007, GM 1500-6TG-2, LICR-LON-HMy2, UC729-6 and the like can be mentioned.

Fusion operation can be performed according to a known method, for example, the method of Koehler and Milstein [Nature, 256, 495 (1975)]. As a fusion promoter, polyethylene glycol (PEG), Sendai virus and the like can be mentioned, and PEG and the like are preferably used. Although the molecular weight of PEG is not subject to limitation, PEG1000 to PEG6000, which are of low toxicity and relatively low viscosity, are preferable. As examples of the PEG concentration, about 10 to 800, preferably about 30 to 50%, can be mentioned. As the solution for diluting PEG, various buffers such as serum-free medium (e.g., RPMI1640), complete medium comprising about 5 to 20% serum, phosphate buffered saline (PBS), and Tris buffer can be used. DMSO (e.g., about 10 to 20%) can also be added as desired. As examples of the pH of the fusion solution, about 4 to 10, preferably about 6 to 8 can be mentioned.

The ratio by number of antibody-producing cells (splenocytes) and myeloma cells is preferably about 1:1 to 20:1, and the cell fusion can be efficiently performed by incubation normally at 20 to 40° C., preferably at 30 to 37° C., normally for 1 to 10 minutes.

An antibody-producing cell line can also be obtained by infecting antibody-producing cells with a virus capable of transforming lymphocytes to immortalize the cells. As such viruses, for example, Epstein-Barr (EB) virus and the like can be mentioned. Although the majority of persons have immunity because they have ever been infected with this virus in an asymptomatic infection of infectious mononucleosis, virion is also produced when the ordinary EB virus is used; therefore, appropriate purification must be performed. As an EB system free from the possibility of viral contamination, it is also preferable to use a recombinant EB virus that retains the capability of immortalizing B lymphocytes but lacks the capability of replicating virion (e.g., deficiency of the switch gene for transition from latent infection state to lytic infection state and the like).

Since marmoset-derived B95-8 cells secrete EB virus, B lymphocytes can be easily transformed by using a culture supernatant thereof. An antibody-producing B cell line can be obtained by, for example, culturing these cells using a medium supplemented with serum and penicillin/streptomycin (P/S) (e.g., RPMI1640) or a serum-free medium supplemented with a cell proliferation factor, thereafter separating the culture supernatant by filtration or centrifugation and the like, suspending therein antibody-producing B lymphocytes at a suitable concentration (e.g., about 10⁷ cells/mL), and incubating the suspension normally at 20 to 40° C., preferably at 30 to 37° C., normally for about 0.5 to 2 hours. When human antibody-producing cells are provided as mixed lymphocytes, it is preferable to previously remove T lymphocytes by allowing them to form an E rosette with, for example, sheep erythrocytes and the like, to increase transformation frequency of EB virus, because the majority of persons have T lymphocytes which exhibit cytotoxicity to cells infected with EB virus. It is also possible to select lymphocytes specific for the target antigen by mixing sheep erythrocytes, previously bound with a soluble antigen, with antibody-producing B lymphocytes, and separating the rosette using a density gradient of percoll and the like. Furthermore, because antigen-specific B lymphocytes are capped by adding the antigen in large excess so that they no longer present IgG to the surface, mixing with sheep erythrocytes bound with anti-IgG antibody results in the formation of rosette only by antigen-nonspecific B lymphocytes. Therefore, by collecting a layer of cells that don't form rosette from this mixture using a density gradient of percoll and the like, it is possible to select antigen-specific B lymphocytes.

Human antibody-secreting cells having acquired the capability of proliferating indefinitely by the transformation can be back fused with mouse or human myeloma cells in order to stably sustain the antibody-secreting ability. As the myeloma cells, the same as those described above can be used.

Hybridoma screening and breeding are normally performed using a medium usable for animal cells (e.g., RPMI1640) comprising 5 to 20% FCS or a serum-free medium supplemented with cell proliferation factors, with the addition of HAT (hypoxanthine, aminopterin, thymidine). As examples of the concentrations of hypoxanthine, aminopterin and thymidine, about 0.1 mM, about 0.4 μM and about 0.016 mM and the like, respectively, can be mentioned. For selecting a human-mouse hybridoma, ouabain resistance can be used. As human cell lines are more susceptible to ouabain than mouse cell lines, it is possible to eliminate unfused human cells by adding ouabain at about 10⁻⁷ to 10⁻³ M to the medium.

In selecting a hybridoma, it is preferable to use feeder cells or culture supernatants of certain cells. As the feeder cells, an allogenic cell species having a lifetime limited so that it dies after helping the emergence of hybridoma, cells capable of producing large amounts of a growth factor useful for the emergence of hybridoma with their proliferation potency reduced by irradiation and the like, and the like are used. For example, as the mouse feeder cells, splenocytes, macrophage, blood, thymocytes and the like can be mentioned; as the human feeder cells, peripheral blood mononuclear cells and the like can be mentioned. As examples of the cell culture supernatant, primary culture supernatants of the above-described various cells and culture supernatants of various established cell lines can be mentioned.

Moreover, a hybridoma can also be selected by reacting a fluorescein-labeled antigen with fusion cells, and thereafter separating the cells that bind to the antigen using a fluorescence-activated cell sorter (FACS). In this case, efforts for cloning can be lessened significantly because a hybridoma that produces an antibody against the target antigen can be directly selected.

For cloning a hybridoma that produces a monoclonal antibody against the target antigen, various methods can be used.

It is preferable to remove aminopterin as soon as possible because it inhibits many cell functions. In the case of mice and rats, aminopterin can be removed 2 weeks after fusion and beyond because most myeloma cells die within 10 to 14 days. However, a human hybridoma is normally maintained in a medium supplemented with aminopterin for about 4 to 6 weeks after fusion. It is desirable that hypoxanthine and thymidine be removed more than one week after the removal of aminopterin. That is, in the case of mouse cells, for example, a complete medium (e.g., RPMI1640 supplemented with 10% FCS) supplemented with hypoxanthine and thymidine (HT) is added or exchanged 7 to 10 days after fusion. About 8 to 14 days after fusion, visible clones emerge. Provided that the diameter of clone has reached about 1 mm, the amount of antibody in the culture supernatant can be measured.

A measurement of the amount of antibody can be performed by, for example, a method comprising adding the hybridoma culture supernatant to a solid phase (e.g., microplate) to which the target antigen or a derivative thereof or its partial peptide (including the partial amino acid sequence used as the epitope) is adsorbed directly or with a carrier, subsequently adding an anti-immunoglobulin (IgG) antibody (an antibody against IgG derived from an animal of the same species as the animal from which the original antibody-producing cells are derived is used) or protein A, which had been labeled with a radioactive substance (e.g., ¹²⁵I, ¹³¹I, ³H, ¹⁴C), enzyme (e.g., β-galactosidase, β-glucosidase, alkaline phosphatase, peroxidase, malate dehydrogenase), fluorescent substance (e.g., fluorescamine, fluorescein isothiocyanate), luminescent substance (e.g., luminol, luminol derivative, luciferin, lucigenin) and the like, and detecting the antibody against the target antigen (epitope) bound to the solid phase, a method comprising adding the hybridoma culture supernatant to a solid phase to which an anti-IgG antibody or protein A is adsorbed, adding the target antigen, a derivative thereof, or its partial peptide labeled with the same labeling reagent as described above, and detecting the antibody against the target antigen (epitope) bound to the solid phase and the like.

Although limiting dilution is normally used as the cloning method, cloning using soft agar and cloning using FACS (described above) are also possible. Cloning by limiting dilution can be performed by, for example, the following procedures, which, however, are not to be construed as limiting.

The amount of antibody is measured as described above, and positive wells are selected. Selected suitable feeder cells are previously added to a 96-well plate. Cells are collected from the antibody-positive wells and suspended in complete medium (e.g., RMPI1640 supplemented with 10% FCS and P/S) to obtain a density of 30 cells/mL; 0.1 mL (3 cells/well) of this suspension is added to the 96-well plate with feeder cells added thereto; a portion of the remaining cell suspension is diluted to 10 cells/mL and sown to other wells (1 cell/well) in the same way; the still remaining cell suspension is diluted to 3 cells/mL and sown to other wells (0.3 cells/well). The cells are cultured for about 2 to 3 weeks until a visible clone appears, when the amount of antibody is measured to select positive wells, and the selected cells are recloned in the same way. In the case of human cells, cloning is relatively difficult, so that a plate in which cells are plated at 10 cells/well is also prepared. Although a monoclonal antibody-producing hybridoma can be obtained normally by two times of subcloning, it is desirable to repeat recloning regularly for several more months to confirm the stability thereof.

Hybridomas can be cultured in vitro or in vivo.

As a method of in vitro culture, a method comprising gradually scaling up a monoclonal antibody-producing hybridoma obtained as described above, from a well plate, while keeping the cell density at, for example, about 10⁵ to 10⁶ cells/mL, and gradually lowering the FCS concentration, can be mentioned.

As a method of in vivo culture, for example, a method comprising an intraperitoneal injection of a mineral oil to a mouse (a mouse that is histocompatible with the parent strain of the hybridoma) to induce plasmacytoma (MOPC) 5 to 10 days later, to which intraperitoneally injecting about 10⁶ to 10⁷ cells of hybridoma, and collecting ascites fluid under anesthesia 2 to 5 weeks later, can be mentioned.

### (b) Purification of the Monoclonal Antibody

Separation and purification of the monoclonal antibody are performed according to a publicly known method such as purification method of immunoglobulin [e.g., salting-out, alcohol precipitation, isoelectric point precipitation, electrophoresis, adsorption-desorption with an ion exchanger (e.g., DEAF, QEAE), ultracentrifugation, gel filtration, specific purification comprising selectively collecting the antibody by means of an antigen-coupled solid phase or an active adsorbent such as protein A or protein G, and dissociating the linkage to obtain the antibody, and the like] in the same manner as an ordinary separation and purification.

As described above, a monoclonal antibody can be produced by culturing, in vivo or in vitro, a hybridoma of a warm-blooded animal, and harvesting an antibody from the body fluid or culture thereof.

When using the antibody of the present invention for prophylaxis or treatment of cancer, since the antibody is required to have antitumor activity, it is necessary to examine the level of antitumor activity of provided monoclonal antibody. The antitumor activity can be assayed by comparing the cancer cell proliferation or induction of apoptosis etc., in the presence or absence of antibody.

An example of the monoclonal antibody of the present invention thus obtained is the mouse anti-human NGF2/NT-3 neutralizing antibody (3W3 antibody) described in JP-A-6-189787. The mouse hybridoma 3W3 cell line, which produces the 3W3 antibody, has been deposited under the accession number FERM BP-3932 at the former National Institute of Bioscience and Human-Technology, Agency of Industrial Science and Technology, Ministry of International Trade and Industry (now the International Patent Organism Depositary, National Institute of Advanced Industrial Science and Technology (IPOD: Chuo 6, Tsukuba Center, 1-1-1, Higashi, Tsukuba, Ibaraki, 305-8566 Japan) since Jul. 15, 1992.

The 3W3 antibody is configured with a heavy chain consisting of the amino acid sequence shown by amino acid numbers 1 to 445 in the amino acid sequence shown by SEQ ID NO:4, and a light chain consisting of amino acid numbers 1 to 218 in the amino acid sequence shown by SEQ ID NO:6.

### (3) Preparation of Chimeric/Humanized/Humanized Antibody

In a preferred mode of embodiment, since the antibody of the present invention is used as a pharmaceutical product having humans as the subject of administration thereof, the antibody used in the present invention (preferably a monoclonal antibody) is an antibody whose risk of showing antigenicity when administered to a human has been reduced; to be specific, the antibody is a fully human antibody, a humanized antibody, a non-human (e.g., mouse)—human chimeric antibody and the like, particularly preferably a fully human antibody. A humanized antibody and a chimeric antibody can be prepared by genetic engineering technology according to the method described below. Although a fully human antibody can also be produced from the above-described human-human (or human-mouse) hybridoma, it is desirable to produce it using a human antibody-producing animal described below (e.g., mouse) or the phage display method in order to stably supply the antibody in large amounts at low costs.

### (i) Preparation of Chimeric Antibody

As used herein, “chimeric antibody” means an antibody wherein the sequences of the variable regions of the H chain and L chain (V_{H} and V_{L}) thereof are derived from a warm-blooded species, and wherein the sequences of the constant regions (C_{H} and C_{L}) are derived from another warm-blooded species. The sequences of the variable regions are preferably derived from, for example, an animal species permitting easy preparation of a hybridoma, such as mouse, and the sequences of the constant regions are preferably derived from the recipient species.

As examples of the method of preparing a chimeric antibody, the method described in U.S. Pat. No. 6,331,415 or a partially modified method thereof and the like can be mentioned. To be specific, first, mRNA or total RNA is prepared from a monoclonal antibody-producing hybridoma (e.g., mouse-mouse hybridoma) obtained as described above, according to a conventional method, to synthesize by a cDNA reverse transcription reaction. DNAs that encode V_{H} and V_{L} are amplified and purified by PCR according to a conventional method with the cDNA as the template, using appropriate primers [for example, oligo DNAs comprising the base sequences that encode the N-terminal sequences of V_{H} and V_{L}, or a signal sequence, respectively, as the sense primers, and oligo DNAs that hybridize to the base sequences that encode J region of each chain, respectively, as the antisense primer (see, for example, Bio/Technology, 9: 88-89, 1991)]. For example, the above-described 3W3 antibody has a heavy chain variable region (V_{H}) consisting of the amino acid sequence shown by amino acid numbers 1 to 121 in the amino acid sequence shown by SEQ ID NO:4, and a light chain variable region (V_{L}) consisting of the amino acid sequence shown by amino acid numbers 1 to 111 in the amino acid sequence shown by SEQ ID NO:6. It is therefore possible to amplify a DNA fragment comprising a base sequence that encodes the V_{H} shown by base numbers 107 to 469 in the base sequence shown by SEQ ID NO:3, and a DNA fragment comprising a base sequence that encodes the V_{L} shown by base numbers 84 to 416 in the base sequence shown by SEQ ID NO:5, by preparing a primer set on the basis of information on the base sequences that encode the heavy and light chains of the 3W3 antibody (base sequences shown by SEQ ID NO:3 and SEQ ID NO:5, respectively), and performing PCR with a cDNA prepared from a 3W3 cell as the template. These DNA fragments may further comprise a base sequence that encodes a secretory signal sequence on the 5′ side thereof. It is preferable to join a restriction endonuclease recognition site for cloning to each end of the DNA fragments.

In the same manner, DNAs that encode C_{H} and C_{L} are amplified and purified from an RNA prepared from lymphocytes and the like of another warm-blooded animal (e.g., human) by RT-PCR. V_{H} and C_{H}, and V_{L} and C_{L}, are ligated together, respectively, using a conventional method, and the chimeric H chain DNA and chimeric L chain DNA obtained are inserted into respective appropriate expression vectors [for example, vectors comprising promoters that have transcription activity in CHO cells, COS cells, mouse myeloma cells and the like (e.g., CMV promoter, SV40 promoter and the like)]. When the chimera heavy chain DNA and chimera light chain DNA do not comprise a base sequence that encodes a secretory signal sequence, the DNAs can be inserted into a secretory expression vector harboring a base sequence that encodes a peptide capable of functioning as a secretion signal in the aforementioned host cell, downstream of the promoter. The DNAs that encode the two chains may be inserted into separate vectors, and may be inserted into a single vector in tandem. It is preferable to utilize as the expression vector, one comprising a previously introduced DNA that encodes the C_{H} or C_{L} derived from a human antibody, such as AG-γ1 comprising a DNA that encodes the constant region of human Igγ1 or AG-κ comprising a DNA that encodes the constant region of human Igκ (see WO 94/20632), because an expression vector harboring the human/non-human chimeric antibody gene of the present invention can be constructed simply by inserting thereinto a DNA that encodes the V_{H} or V_{L} of a non-human antibody.

Host cells are transformed with the chimeric H chain and chimeric L chain expression vector(s) obtained. As the host cells, animal cells, for example, Chinese hamster ovary (CHO) cells, monkey-derived COS-7 cells, Vero cells, rat-derived GHS cells and the like, in addition to the above-described mouse myeloma cells, can be mentioned. For the transformation, any method applicable to animal cells can be used, with preference given to electroporation method and the like. It is possible to isolate a chimeric monoclonal antibody by culturing the host cells in a medium suitable thereto for a given period, and thereafter collecting the culture supernatant and purifying it in the same manner as described above. Alternatively, it is also possible to obtain a chimeric monoclonal antibody easily and in large amounts from milk or eggs of transgenic animals which are produced by a conventional method using germ line cells of an animal such as bovine, goat, or fowl as the host cells, for which a transgenic technique has been established and a know-how of mass propagation as a domestic animal (domestic fowl) has been compiled. Furthermore, it is also possible to obtain a chimeric monoclonal antibody in large amounts from the seeds, leaves and the like of a transgenic plant, produced by using microinjection and electroporation into protoplast, the particle gun method and Ti-vector method for intact cells and the like, with cells of a plant such as corn, rice, wheat, soybean, or tobacco as the host cells, for which a transgenic technique has been established, and which is cultured in large amounts as a major crop.

When the obtained chimeric monoclonal antibody is digested with papain, Fab is obtained; when the same is digested with pepsin, F(ab′)₂ is obtained.

It is also possible to reformat into scFv by ligating DNAs that encode mouse V_{H} and V_{L} via a suitable linker, for example, a DNA that encodes a peptide consisting of 1 to 40 amino acids, preferably 3 to 30 amino acids, more preferably 5 to 20 amino acids [e.g., [Ser-(Gly)ₘ]ₙ or [(Gly)ₘ-Ser]ₙ (m is an integer from 0 to 10, n is an integer from 1 to 5) and the like]. Furthermore, it is possible to reformat into a minibody by ligating a DNA that encodes C_{H3} via a suitable linker thereto, or reformat into a scFv-Fc by ligating a DNA that encodes C_{H} full length via a suitable linker thereto. The DNA encoding such an antibody molecule modified (coupled) by genetic engineering can be expressed in a microorganism such as E. coli or yeast under the control of a suitable promoter, to produce the antibody molecule in large amounts.

When DNAs encoding mouse V_{H} and V_{L} are inserted into the downstream of one promoter in tandem and introduced into E. coli, a dimer named as Fv is formed by monocistronic gene expression. When appropriate amino acids in the FRs of V_{H} and V_{L} are substituted with Cys using molecular modeling, a dimer named as dsFv is formed via the intermolecular disulfide bond between the two chains.

### (ii) Humanized Antibody

As used herein, “a humanized antibody” means an antibody wherein the sequences of all regions present in the variable region, other than the complementality determining region (CDR), [i.e., framework region (FR) in constant region and variable region] are derived from a human, and wherein only the sequence of CDR is derived from another mammalian species. The other mammalian species is preferably an animal species, for example, mouse and the like, with which production of hybridomas can be easily performed.

As examples of the method of preparing a humanized antibody, the methods described in U.S. Pat. Nos. 5,225,539, 5,585,089, 5,693,761 and 5,693,762 or partially modified methods therefrom and the like can be mentioned. To be specific, DNAs that encode V_{H} and V_{L} derived from a non-human mammalian species (e.g., mouse) are isolated in the same manner as with the above-described chimeric antibody, after which sequencing is performed by a conventional method using an automated DNA sequencer (e.g., manufactured by Applied Biosystems Company and the like), and the base sequences obtained or deduced amino acid sequences therefrom are analyzed using a known antibody sequence database [for example, Kabat database (see Kabat et al., “Sequences of Proteins of Immunological Interest”, edited by NIH, US Department of Health and Human Services, Public Health Service, 5th edition, 1991) and the like] to determine the CDR and FR of the two chains. In the case of 3W3 antibody, for example, heavy chain CDR is assumed to be amino acid Nos. 26-35 (CDR-H1), 50-66 (CDR-H2) and 102-110 (CDR-H3) of the amino acid sequence shown by SEQ ID NO: 4, and light chain CDR is assumed to be amino acid Nos. 24-38 (CDR-L1), 54-60 (CDR-L2) and 93-100 (CDR-H3) of the amino acid sequence shown by SEQ ID NO:6. A base sequence wherein the CDR encoding region of a base sequence that encodes the L chain and H chain of a human antibody having an FR sequence similar to the determined FR sequence [e.g., human κ type L chain subgroup I and human H chain subgroup II or III (see Kabat et al., 1991 (supra))] is substituted with the determined base sequence that encodes the CDR of another animal species, is designed, and the base sequence is divided into fragments of about 20 to 40 bases, and a sequence complementary to the base sequence is divided into fragments of about 20 to 40 bases so that they alternatively overlap with the aforementioned fragments. It is possible to construct DNAs that encode V_{H} and V_{L} having human-derived FR and a CDR derived from another mammalian species by synthesizing individual fragments using a DNA synthesizer, and hybridizing and ligating them in accordance with conventional methods. In order to transfer a CDR derived from another mammalian species into human-derived V_{H} and V_{L} more quickly and more efficiently, it is preferable to use PCR-based site directed mutagenesis. As examples of such a method, the sequential CDR grafting method described in JP-A-5-227970 and the like can be mentioned. Alternatively, entire DNAs encoding humanized antibodies V_{H} and V_{L} may be chemically synthesized.

It should be noted that in preparing a humanized antibody by a method as described above, the antigen binding activity may sometimes decrease, compared with the original non-human antibody, if the amino acid sequence of CDR alone is transplanted to the template human antibody FR. In such cases, it is effective to transplant some FR amino acids around the CDR in combination. The non-human antibody FR amino acid to be transplanted may be an amino acid residue that is important to the maintenance of the steric structure of each CDR; such an amino acid residue can be deduced by a steric structure estimation using a computer.

It is possible to obtain cells or transgenic animal/plant that produces a humanized antibody by ligating the thus-obtained DNAs encoding V_{H} and V_{L} to DNAs encoding human-derived C_{H} and C_{L}, respectively, in the same manner as with the above-described chimeric antibody, and introducing the ligated product into suitable host cells, or by synthesizing the entire humanized antibody genes and introducing them into suitable host cells.

An alternative method of preparing a humanized antibody without using CDR grafting is, for example, a method comprising determining which is an amino acid residue that can be substituted in a non-human variable region, on the basis of a conserved structure-function correlation between antibodies. This method can be carried out as described in, for example, EP 0571613 B1, U.S. Pat. No. 5,766,886, U.S. Pat. No. 5,770,196, U.S. Pat. No. 5,821,123, U.S. Pat. No. 5,869,619 and the like. Provided that the amino acid sequence information on each of the V_{H} and V_{L} of the original non-human antibody, preparation of a humanized antibody using the method can easily be performed by utilizing, for example, the contract antibody preparation service provided by Xoma.

A humanized antibody, like a chimeric antibody, can be modified to scFv, scFv-Fc, minibody, dsFv, Fv and the like by using genetic engineering techniques; and they can be produced in a microorganism such as E. coli or yeast by using a suitable promoter.

The technology for preparing a humanized antibody can also be applied to, for example, preparing a monoclonal antibody that can be preferably administered to another animal species for which no hybridoma production technology has been established. For example, animals widely propagated as domestic animals (domestic fowl) such as bovine, swine, sheep, goat, and fowl, and pet animals such as dog and cat, and the like can be mentioned as the subject animal species.

(iii) Preparation of Fully Human Antibody Using Human Antibody-Producing Animal

Provided that a functional human Ig gene is introduced into a non-human warm-blooded animal having the endogenous immunoglobulin (Ig) gene knocked out (KO) therein, and that this animal is immunized with an antigen, a human antibody is produced in place of the antibody derived from the animal. Therefore, provided that an animal such as mice, for which a technique for producing a hybridoma has been established, is used, it is possible to acquire a fully human monoclonal antibody by the same method as the conventional method used to prepare a mouse monoclonal antibody. First, some of the human monoclonal antibodies, that were generated by using a human antibody-producing mouse (see Immunol. Today, 17: 391-397, 1996) obtained by crossing a mouse transfected with minigenes of the human Ig H chain and L chain using an ordinary transgenic (Tg) technique with a mouse wherein the endogenous mouse Ig gene has been inactivated using an ordinary KO technique, are already in clinical stage, and to date production of anti-human Ig human antibody (HAHA) has not been reported.

Later, Abgenix Inc. [trade name: XenoMouse (see Nat. Genet., 15: 146-156, 1997; U.S. Pat. No. 5,939,598 and the like)] and Medarex Inc. [trade name: Hu-Mab Mouse (see Nat. Biotechnol., 14: 845-851, 1996; U.S. Pat. No. 5,545,806 and the like)] established Tg mice transfected with even a larger human Ig gene using a yeast artificial chromosome (YAC) vector, thus enabling the production of human antibodies of richer repertoire. However, because the human Ig gene, for example, in the case of the H chain, exhibits its diversity as the VDJ exon, which is a variable combination of about 80 kinds of V fragments, about 30 kinds of D fragments and 6 kinds of J fragments, encodes the antigen binding site, the full length thereof is as large as about 1.5 Mb (14th chromosome) for the H chain, about 2 Mb (2nd chromosome) for the KL chain, and about 1 Mb (22nd chromosome) for the AL chain. To reproduce the diverse antibody repertoire in human in another animal species, it is desirable to introduce the full length of each Ig gene. However, since a DNA that is insertable into a conventional transfection vector (plasmid, cosmid, BAC, YAC and the like) is normally several kb to several hundred kb in length, it has been difficult to introduce the full length of Ig genes by the conventional technique for establishing a transgenic animal, which comprises inserting a cloned DNA into a fertilized egg.

Tomizuka et al. (Nat. Genet., 16: 133-143, 1997) prepared a mouse having the full-length human Ig gene by introducing a natural fragment of a human chromosome harboring the Ig gene (hCF) into a mouse [transchromosomic (TC) mouse]. That is, first, a human-mouse hybrid cell having human chromosomes in which the 14th chromosome comprising the H chain gene and the 2nd chromosome comprising the KL chain gene, both labeled with, for example, a drug-resistance marker and the like, is treated with a spindle formation inhibitor (e.g., colcemid) for about 48 hours to prepare a microcell wherein one to several chromosomes or fragments thereof are enveloped in nuclear membrane, and the chromosomes are introduced into a mouse ES cell by the micronuclear fusion method. A hybrid ES cell retaining the chromosomes having the human Ig gene or fragments thereof is selected using a medium comprising a drug, and the cell is microinjected into a mouse embryo in the same manner as with the preparation of an ordinary KO mouse. A germ line chimera is selected among the chimeric mice obtained, with coat color as the index, and the like, to establish a TC mouse strain carrying the human 14th chromosome fragment (TC(hCF14)) and a TC mouse strain carrying the human 2nd chromosome fragment (TC(hCF2)). After establishing mouse strains wherein the endogenous H chain gene and κL chain gene are knocked out, respectively [KO (IgH) and KO (Igκ)] by a conventional method, it is possible to establish a mouse strain having all the four kinds of gene modifications (double TC/KO) by repeating the crossing of these four strains.

Provided that the same method as that for producing an ordinary mouse monoclonal antibody is applied to a double TC/KO mouse established as described above, it is possible to obtain an antigen-specific human monoclonal antibody-producing hybridoma. However, there is the drawback of a lower efficiency to obtain hybridomas than that with the ordinary mouse, because hCF2 comprising the KL chain gene is unstable in the mouse cells.

On the other hand, because the aforementioned Hu-Mab mouse has a structure wherein the variable region cluster are doubled although it has about 50% of the KL chain gene, it exhibits a K chain diversity equivalent to that with full length (on the other hand, HuMab mouse exhibits a low H chain diversity and inadequate response to antigen because it carries only about 10% of the H chain gene). And the κ chain is stably retained in the mouse cells because it is inserted in mouse chromosome via a YAC vector (Igκ-YAC). Making use of this advantage, it is possible to get the efficiency for obtaining hybridomas and affinity to antigen affinity of antibody that are equivalent to those with the ordinary mouse, by crossing a TC(hCF14) mouse with a Hu-Mab mouse to establish a mouse that stably retains both hCF14 and Igκ-YAC (trade name: KM mouse).

Furthermore, it is also possible to establish a human antibody-producing animal in which the λL chain gene is further transfected to reconstruct the diverse human antibody repertoire more completely. Such an animal can also be obtained by producing a TC mouse in which the human 22nd chromosome or a fragment thereof harboring the λL chain gene is introduced in the same manner as described above [TC(hCF22)], and crossing the mouse with the above-described double TC/KO mouse or KM mouse, or can also be obtained by, for example, constructing a human artificial chromosome (HAC) comprising both the H chain locus and the λL chain locus, and introducing it into a mouse cell (Nat. Biotechnol., 18: 1086-1090, 2000).

When an antibody of the present invention is used as a medicine, an antibody of the present invention is desirably a monoclonal antibody, but it may be a polyclonal antibody. When the antibody of the present invention is a polyclonal antibody, it is not necessary to use hybridoma; therefore, provided that a human antibody-producing animal is produced in the same manner as described above using an animal species for which no technique for preparing a hybridoma has been established but a transgenic technique has been established, preferably an ungulate such as bovine, it is also possible to produce a human antibody in larger amounts at low costs (see, for example, Nat. Biotechnol., 20: 889-894, 2002). The human polyclonal antibody thus obtained can be purified by collecting blood, ascites fluid, milk, egg and the like, preferably milk or egg, of the human antibody-producing animal, in combination with the same purification techniques as described above.

### (iv) Preparation of Fully Human Antibody Using Phage Display Human Antibody Library

Another approach to produce a fully human antibody is a method using phage display. This method sometimes encounters cases in which a mutation due to PCR is introduced into a site other than CDRs; for this reason, a few reports of cases of HAHA production in clinical stage are available. On the other hand, however, the method has advantages such as no risk of cross-species viral infection derived from the host animal and the indefinite specificity of the antibody (antibodies against forbidden clone, sugar chain and the like can also be easily prepared).

The method of preparing a phage display human antibody library include, but are not limited to, for example, the methods described below.

Although a phage used is not subject to limitation, filamentous phage (Ff bacteriophage) is normally preferably used. As the method of presenting a foreign protein on the phage surface, a method comprising expressing and presenting the foreign protein as a fusion protein with any of the coat proteins g3p, and g6p to g9p on the coat protein can be mentioned; and a method comprising fusing the foreign protein to the N-terminal side of g3p or g8p is often used. As the phage display vector, besides 1) one in which the foreign gene is introduced in the form of fusion gene with the coat protein gene of the phage genome, to allow all the coat proteins presented on the phage surface to be presented as a fusion protein with the foreign protein, 2) one in which the gene encoding the fusion protein is inserted separately from the wild-type coat protein gene to allow the fusion protein and the wild-type coat protein to be expressed simultaneously, and 3) an E. coli having a phagemid vector harboring the gene that encodes the fusion protein is infected with a helper phage having the wild-type coat protein gene to produce phage particles that express the fusion protein and the wild-type coat protein simultaneously, and the like can be mentioned. However, a phage display vector of the type 2) or 3) is used for the preparation of an antibody library, because in the case of 1), the capability of infection is lost when a large foreign protein is fused.

As a specific vector, those described by Holt et al. (Curr. Opin. Biotechnol., 11: 445-449, 2000) can be mentioned as examples. For example, pCES1 (see J. Biol. Chem., 274: 18218-18230, 1999) is an Fab-expressing phagemid vector wherein a DNA encoding the κL chain constant region allocated to downstream of the g3p signal peptide, and a DNA encoding CH3, His-tag, c-myc tag, and the amber stop codon (TAG) followed by the g3p coding sequence, allocated to downstream of the g3p signal peptide, are arranged under the control of one lactose promoter. When this is introduced to an E. coli having an amber mutation, Fab is presented onto the g3p coat protein, but when it is expressed in the HB2151 strain and the like, which do not have an amber mutation, a soluble Fab antibody is produced. As the scFv-expressing phagemid vector, for example, pHEN1 (J. Mol. Biol., 222: 581-597, 1991) and the like are used.

Meanwhile as examples of the helper phage, M13-KO7, VCSM13 and the like can be mentioned.

And as another phage display vector, a vector that is designed as a DNA sequence comprising the cysteine-encoding codon is linked to each of the 3′ end of the antibody gene and the 5′ end of the coat protein gene to express the two genes simultaneously and separately (not in the form of a fusion protein), and to present the antibody onto the coat protein on the phage surface via S-S bonds between the introduced cysteine residues (CysDisplay™ technology of Morphosys Company) and the like, can be mentioned.

As the kind of human antibody library, a naive/non-immunized library, a synthetic library, an immunized library and the like can be mentioned.

The naive/non-immunized library is a library obtained by acquiring the V_{H} and V_{L} genes retained by a normal human by RT-PCR, and randomly cloning them into the above-described phage display vector. Normally, mRNA derived from lymphocytes of peripheral blood, bone marrow, tonsil and the like of a normal human, and the like are used as the template. A library prepared by selectively amplifying IgM-derived mRNA in which a class switch due to antigen sensitization is not undergoing, to avoid V gene biases such as clinical history, is particularly called a naive library. Representatively, the library of Cambridge Antibody Technology (see J. Mol. Biol., 222: 581-597, 1991; Nat. Biotechnol., 14: 309-314, 1996), the library of Medical Research Council (see Annu. Rev. Immunol., 12: 433-455, 1994), the library of Dyax Corp. (see J. Biol. Chem., 1999 (supra); Proc. Natl. Acad. Sci. USA, 14: 7969-7974, 2000) and the like can be mentioned.

A synthetic library is obtained by selecting a functional particular antibody gene in human B cells, and substituting a portion of antigen-binding region in a V gene segment, for example, CDR3 and the like, with DNAs encoding a random amino acid sequence of appropriate length, to construct a library. It is recognized to be excellent in antibody expression efficiency and stability because the library can be constructed with the combination of the V_{H} and V_{L} genes, which produce functional scFv and Fab, since the beginning. Representatively, the HuCAL library of Morphosys AG (see J. Mol. Biol., 296: 57-86, 2000), the library of BioInvent (see Nat. Biotechnol., 18: 852, 2000), the library of Crucell (see Proc. Natl. Acad. Sci. USA, 92: 3938, 1995; J. Immunol. Methods, 272: 219-233, 2003) and the like can be mentioned.

An immunized library is a library obtained by preparing an mRNA from lymphocytes collected from a human such as a patient with cancer, autoimmune disease, infectious disease and the like or a recipient of vaccination, having an elevated blood antibody titer against the target antigen, or from human lymphocytes and the like which are artificially immunized with the target antigen by the above-described in vitro immunization method, in the same manner as with the above-described naive/non-immunized library, and amplifying the V_{H} and V_{L} genes by RT-PCR, to construct a library. It is possible to obtain the desired antibody even from such libraries of relatively small size because the desired antibody gene is contained in the library already at the beginning.

The wider the diversity of the library is, the better; actually, however, an appropriate library size is about 10⁸ to 10¹¹ clones, taking into consideration of the number of phages handlable in the following panning operation (10¹¹ to 10¹³ phages) and the number of phages necessary to isolate and amplify clones in ordinary panning (100 to 1,000 phages/clone), it is possible to screen for an antibody normally having a Kd value on the order of 10⁻⁹ with a library of about 10⁸ clones.

The process for selecting an antibody against the target antigen by the phage display method is referred to as panning. To be specific, for example, a phage presenting an antigen-specific antibody is concentrated by repeating a series of operations of bringing an antigen-immobilized carrier and a phage library into contact with each other, washing out the unbound phage, thereafter eluting the bound phage from the carrier, and infecting the phage to E. coli to proliferate it, about 3 to 5 times. As the carrier for immobilizing the antigen, various carriers used in ordinary antigen-antibody reactions or affinity chromatography, for example, insoluble polysaccharides such as agarose, dextran, and cellulose, synthetic resins such as polystyrene, polyacrylamide, and silicon, or microplates, tubes, membranes, columns, beads and the like comprising glass, metal and the like, and surface plasmon resonance (SPR) sensor chips, and the like can be mentioned. For the antigen immobilization, physical adsorption may be used, and a method using a chemical bond used to insolubilize and immobilize a protein or enzyme and the like is also acceptable. For example, a biotin-(strept)avidin, system and the like are preferably used. When the endogenous ligand, that is a target antigen, is a small molecule such as a peptide, it is necessary to pay special attention to prevent masking of the portion used as the epitope by conjugating with the carrier. For washing the unbound phage, a blocking solution such as BSA solution (once or twice), a PBS comprising a surfactant such as Tween (3 to 5 times) and the like can be used. There is also a report mentioning that the use of citrate buffer (pH 5) and the like is preferable for the washing. For elution of the specific phage, an acid (e.g., 0.1 M hydrogen chloride and the like) is normally used; cleavage with a specific protease (e.g., a gene sequence that encodes the trypsin cleavage site can be introduced into the linkage site between the antibody gene and the coat protein gene. In this case, E. coli infection and proliferation are possible even if all the coat protein is expressed in the form of a fusion protein because the wild-type coat protein is presented on the surface of the eluted phage), competitive elution with a soluble antigen, or elution by reduction of S-S bond (e.g., in the aforementioned CysDisplay™, the antigen-specific phage can be collected by dissociating the antibody and the coat protein by using a suitable reducing agent after performing panning.) is also possible. When elution has been performed with an acid, the eluate is neutralized with Tris and the like, and the eluted phage is then infected to E. coli, which is cultured; after which the phage is collected by a conventional method.

After the phage presenting the antigen-specific antibody is concentrated by panning, the phage is infected to E. coli and the cells are sown onto a plate to perform cell cloning. The phage is again collected from the each clone, and the antigen binding activity is confirmed by the above-described antibody titer assay (e.g., ELISA, RIA, FIA and the like) or a measurement utilizing FACS or SPR.

Isolation and purification of the antibody from the selected phage clone that presents the antigen-specific antibody can be performed by, for example, when using a vector incorporating an amber stop codon at the linker site of the antibody gene and the coat protein gene as the phage display vector, infecting the phage to an E. coli that does not have amber mutation (e.g., HB2151 strain) to produce and secrete soluble antibody molecules in periplasm or the medium, lysing the cell wall with lysozyme and the like, collecting the extracellular fraction, and purifying using the same purification technique as described above. Provided that the His-tag or c-myc tag has been introduced in advance, the antibody can easily be purified by using IMAC, an anti-c-myc antibody column and the like. When cleavage with a specific protease is utilized in panning, the antibody molecule is separated from the phage surface by an action with the protease, so that the desired antibody can be purified by performing the same purification operation as above mentioned.

The technology for producing a fully human antibody using a human antibody-producing animal and a phage display human antibody library can also be applied to the production of a monoclonal antibody derived from another animal species. For example, animals widely propagated as domestic animals (domestic fowl) such as bovine, swine, sheep, goat, and fowl, and pet animals such as dog and cat, and the like can be mentioned as the subject animal species. In non-human animals, the utilization of an immunized library is more effective because there are fewer ethical problems concerning artificial immunization with the target antigen.

### (4) Preparation of Polyclonal Antibody

The polyclonal antibody of the present invention can be manufactured by publicly known methods or modifications thereof. For example, a warm-blooded animal is immunized with an immunogen (protein or peptide antigen) per se, or with a complex of immunogen and a carrier protein formed in a manner similar to the method described above for the manufacture of monoclonal antibodies. The product comprising the antibody of the present invention is collected from the immunized animal followed by separation and purification of the antibody.

In the complex of immunogen and carrier protein used to immunize a warm-blooded animal, the type of carrier protein and the mixing ratio of carrier protein to hapten may be any type and in any ratio, as long as the antibody is efficiently produced to the hapten immunized by crosslinking to the carrier protein. For example, bovine serum albumin, bovine thyroglobulin or hemocyanin is coupled to hapten in a carrier-to-hapten weight ratio of approximately 0.1 to 20, preferably about 1 to 5.

A variety of condensation agents can be used for the coupling of carrier protein to hapten. Glutaraldehyde, carbodiimide, maleimide activated ester and activated ester reagents comprising thiol group or dithiopyridyl group are used for the coupling.

The condensation product is administered to warm-blooded animals either solely or together with carriers or diluents to the site that can produce the antibody by the administration. In order to potentiate the antibody productivity upon the administration, complete Freund's adjuvant or incomplete Freund's adjuvant may be administered. The administration is usually made once every about 1 to 6 weeks and about 2 to 10 times in total.

The polyclonal antibody can be collected from the blood, ascites, etc., preferably from the blood of warm-blooded animal immunized by the method described above.

The polyclonal antibody titer in antiserum can be assayed, by the same procedure as the assay of antibody titer of the antiserum described above. The separation and purification of the polyclonal antibody can be carried out, following the method for the separation and purification of immunoglobulins performed as in the separation and purification of monoclonal antibodies described hereinabove.

### (5) Preparation of Fusion or Modified Antibody

Those skilled in the art can prepare a fusion antibody of any one of the above-described antibodies (including antibody fragments) and another peptide or protein and prepare a modified antibody coupled with a modifier on the basis of an obvious technology. The other peptide or protein used for the fusion is not subject to limitations, as far as it does not reduce the binding activity of the antibody; examples include human serum albumin, various tag peptides, artificial helix motif peptide, maltose-binding protein, glutathione S transferase, and various toxins, as well as peptides or proteins capable of promoting polymerization and the like. The modifier used for the modification is not subject to limitations, as far as it does not reduce the binding activity of the antibody; examples include polyethylene glycol, sugar chains, phospholipids, liposome, low-molecular compounds and the like.

### Pharmaceutical Uses for Anti-NGF2/NT-3 Neutralizing Antibody

The antibody of the present invention can be used as an agent for the prophylaxis or treatment of kidney cancer and/or urinary bladder cancer because of the suppressive action thereof on the growth of these cancers. The antibody of the present invention neutralizes NGF2/NT-3. Here, “to neutralize NGF2/NT-3” includes, but is not limited to, the inhibition of signal transduction by the interaction of NGF2/NT-3 with the receptor Trk family proteins and p75NGFR, and is defined as a concept encompassing antibody-dependent cytotoxic activity (ADCC activity), complement-dependent cytotoxic activity (CDC activity), cancer cell proliferation inhibition, apoptosis induction and the like.

The antibody of the present invention suppresses the growth of kidney cancer and urinary bladder cancer. Furthermore, the antibody can also be used to prevent and/or treat other cancers wherein NGF2/NT-3 and/or the receptor Trk family proteins and p75NGFR are expressed because the antibody specifically recognizes NGF2/NT-3 to block (neutralize) the activity thereof. Accordingly, the present invention also provides a prophylactic/therapeutic agent for cancers that produce NGF2/NT-3 and/or Trk family proteins and p75NGFR, comprising the antibody of the present invention.

Examples of cancers that produce NGF2/NT-3 and/or Trk family proteins and p75NGFR include kidney cancer and urinary bladder cancer, as well as, for example, pancreatic cancer, prostate cancer, lung cancer, ovarian cancer, melanoma and the like.

The antibody of the present invention is low-toxic and can be administered as it is in the form of liquid preparations, or as pharmaceutical compositions of suitable preparations to mammals (e.g., human, rats, rabbits, sheep, swines, bovines, cats, dogs, monkeys, etc.) orally or parenterally.

The antibody of the present invention may be administered in itself, or may be administered as an appropriate pharmaceutical composition. The pharmaceutical composition used for the administration may contain a pharmacologically acceptable carrier with the antibody of the present invention, a diluent or excipient. Such a pharmaceutical composition is provided in the form of a pharmaceutical composition (preparation) suitable for oral or parenteral administration. While the content of the antibody in the pharmaceutical composition varies depending on the dosage form, dose and the like, it is, for example, about 0.1-100 wt %.

Examples of the pharmaceutical composition for parenteral administration are injectable preparations, suppositories, etc. The injectable preparations may include dosage forms such as intravenous, subcutaneous, intracutaneous and intramuscular injections, drip infusions, intraarticular injections, etc. These injectable preparations may be prepared by methods publicly known. For example, the injectable preparations may be prepared by dissolving, suspending or emulsifying the antibody of the present invention in a sterile aqueous medium or an oily medium conventionally used for injections. As the aqueous medium for injections, there are, for example, physiological saline, an isotonic solution comprising glucose and other auxiliary agents, etc., which may be used in combination with an appropriate solubilizing agent such as an alcohol (e.g., ethanol), a polyalcohol (e.g., propylene glycol, polyethylene glycol), a nonionic surfactant [e.g., polysorbate 80, HCO-50 (polyoxyethylene (50 mol) adduct of hydrogenated castor oil)], etc. As the oily medium, there are employed, e.g., sesame oil, soybean oil, etc., which may be used in combination with a solubilizing agent such as benzyl benzoate, benzyl alcohol, etc. Thus prepared injectable preparations are subjected, for example, to a sterilization treatment as necessary, such as sterilization by filtration using a membrane filter etc., and preferably filled in a suitable ampoule.

The injectable preparation can also be used as a fresh supply obtained by dissolving (dispersing) a powder prepared by treating the above-described liquid by vacuum drying and the like. As examples of the vacuum drying method, lyophilization, a method using the Speedback Concentrator (SAVANT Company), and the like can be mentioned. When performing lyophilization, it is preferable to lyophilize the sample, cooled to not more than −10° C., using a flask in the laboratory or a tray or vial in industrial settings. When the Speedback Concentrator is used, lyophilization is performed at about 0 to 30° C. under a vacuum of about 20 mmHg or less, preferably about 10 mmHg or less. It is preferable to add a buffering agent such as a phosphate to the liquid to be dried, to obtain a pH of about 3 to 10. The powder preparation obtained by lyophilization, as a long-stable preparation, can be prepared freshly as an injectable preparation by dissolving in water for injection, saline, Ringer's solution and the like, or by dispersing in olive oil, sesame oil, cottonseed oil, corn oil, propylene glycol and the like before use.

The suppository used for rectal administration may be prepared by blending the antibody or the salts thereof described above with conventional bases for suppositories.

For example, the pharmaceutical composition for oral administration includes solid or liquid preparations, specifically, tablets (including dragees and film-coated tablets), pills, granules, powdery preparations, capsules (including soft capsules), syrup, emulsions, suspensions, etc. Such pharmaceutical composition is manufactured by publicly known methods and contains a vehicle, a diluent or excipient conventionally used in the field of pharmaceutical preparations. Examples of the vehicle or excipient for tablets are lactose, starch, sucrose, magnesium stearate, etc.

Advantageously, the pharmaceutical composition for oral or parenteral administration described above is prepared into pharmaceutical preparations with a unit dose suited to fit a dose of the active ingredients. Such unit dose preparations include, for example, tablets, pills, capsules, injections (ampoules), suppositories, etc. The amount of the antibody of the present invention contained is generally 2 to 2000 mg per unit dose preparation; it is preferred that the antibody is contained in the amount of about 2 to about 2000 mg especially in the injections, and in the amount of 2 to 2000 mg in the other unit dose preparations.

The above-described pharmaceutical composition can also be enclosed in a liposome to facilitate the transportation thereof into cells as required. Preferable liposomes include positively charged liposomes, positively charged cholesterols, transmembrane peptide binding liposomes and the like (Mamoru Nakanishi et al., Protein, Nucleic Acid and Enzyme, 44: 1590-1596 (1999), Shiroh Futaki, Kagaku To Seibutsu, 43: 649-653 (2005), Clinical Cancer Research 59: 4325-4333 (1999) and the like).

The administration can be performed parenterally (e.g., intravenous, intra-arterial, intraperitoneal, intramuscular, subcutaneous, intradermal, transdermal, and transmucosal administration and the like) or orally, preferably by intravenous, intraperitoneal, intramuscular, subcutaneous, transdermal, and transmucosal administration and the like.

The dose of the agent described above comprising the antibody of the present invention and the dose of the antibody of the present invention may vary depending upon subject to be administered, target disease, conditions, route of administration, etc. For example, when used for the purpose of treating/preventing kidney cancer or urinary bladder cancer in adult, it is advantageous to administer the antibody intravenously in a dose of about 0.05 to about 50 mg/kg body weight, preferably about 0.125 to about 20 mg/kg body weight and more preferably about 0.25 to about 10 mg/kg body weight. In other parenteral and oral administration, the agent can be administered in a dose corresponding to the dose given above. When the condition is especially severe, the dose may be increased according to the condition. The frequency of administration (period) is, for example, but is not limited to, several times (e.g. 2 or 3 times) at once in 1 or 2 weeks, or once in 2 or 3 weeks for about 2 months and the like.

Each pharmaceutical composition described above may further contain other active components unless formulation causes any adverse interaction with the antibody described above.

Furthermore, the antibody of the present invention may be used in combination with other drugs (also referred to as concomitant drugs), for example, alkylating agents (e.g., cyclophosphamide, ifosfamide, etc.), metabolic antagonists (e.g., methotrexate, 5-fluorouracil, etc.), antitumor antibiotics (e.g., mitomycin, adriamycin, etc.), plant-derived antitumor agents (e.g., vincristine, vindesine, taxol, etc.), cisplatin, carboplatin, etoposide, irinotecan etc. The antibody of the present invention and the concomitant drug described above may be administered simultaneously or at staggered times and simultaneously or at staggered routes to the patient.

The antibody of the present invention can be administered in a state coupled with the above-described concomitant drug as required. The antibody of the present invention transports the concomitant drug to a target disease site where NGF2/NT-3 is present or the vicinity thereof, and inhibits the function of NGF2/NT-3, whereas the concomitant drug treats, mitigates or ameliorates symptoms of the disease.

Coupling of the antibody and the concomitant drug is preferably performed via a linker. The linker is exemplified by one comprising a substituted or unsubstituted aliphatic alkylene chain, and having at both ends thereof a group bindable to a functional group of the antibody or concomitant drug, for example, an N-hydroxysuccinimide group, an ester group, a thiol group, an imidecarbonate group, an aldehyde group or the like (Koutai Kogaku Nyumon, Chijin Shokan, 1994).

### Diagnostic Uses for Anti-Human NGF2/NT-3 Antibody

The present invention also provides a diagnostic reagent of kidney cancer or urinary bladder cancer comprising the antibody of the present invention.

The antibody of the present invention can be used for quantitation of NGF2/NT-3 in test solutions and the like because of its capability of specifically recognizing NGF2/NT-3. Therefore, by measuring the amount of NGF2/NT-3 in a biological sample (e.g., blood, plasma, urine, biopsy sample culture supernatant and the like) from a subject mammal using the antibody of the present invention, the degree of expression of NGF2/NT-3 in the body of the animal can be examined, and hence can be used for the diagnosis of cancer. The NGF2/NT-3 in test solutions can be specifically quantitated by, for example,

(i) a method comprising competitively reacting the antibody of the present invention, a sample fluid, and a labeled form of the NGF2/NT-3, and determining the labeled NGF2/NT-3 that binds to the antibody, thereby to quantify the NGF2/NT-3 in the sample fluid; and
(ii) a method comprising simultaneously or continuously reacting a sample fluid, the immobilized antibody of the present invention on a carrier, and a labeled form of the antibody of the present invention, and measuring the amount (activity) of the label on the immobilizing carrier, thereby to quantify the NGF2/NT-3 in the sample fluid can be mentioned. The immobilized antibody and the labeled antibody may be the same or different.

In the quantification method of (ii) above, two species of antibodies may be the ones that each recognize the different part in NGF2/NT-3. For example, when one antibody recognizes the N-terminal region of the NGF2/NT-3, another antibody recognizing the C-terminal region may be used.

Examples of labeling agents, which are employed for the labeled forms in the aforesaid measuring methods, are radioisotopes, enzymes, fluorescent substances, luminescent substances, etc. Examples of radioisotopes are [¹²⁵I], [¹³¹I], [³H], [¹⁴C], etc. Preferred examples of the enzymes are those that are stable and have a higher specific activity, which include β-galactosidase, β-glucosidase, alkaline phosphatase, peroxidase, malate dehydrogenase, etc. Examples of the fluorescent substances include fluorescamine, fluorescein isothiocyanate, etc. Examples of the luminescent substances are luminol, a luminol derivative, luciferin, lucigenin, etc. Furthermore, a biotin-(strepto)avidin system may be used as well for binding an antibody or antigen to a labeling agent.

As the testsolution, a biological sample (e.g., blood, plasma, urine, biopsy sample culture supernatant and the like) collected from a subject mammal can be used. The biological sample for the diagnosis of kidney cancer or urinary bladder cancer is preferably, for example, urine or a biopsy sample culture supernatant from the target organ, with greater preference given to urine because of the low invasiveness thereof.

The quantification method of the NGF2/NT-3 using the antibody of the present invention is not particularly limited. Any quantification method may be used, so long as the amount of an antibody, antigen or antibody-antigen complex corresponding to the amount of antigen in a sample fluid can be detected by chemical or physical means and the amount of the antigen can be calculated from a standard curve prepared from standard solutions comprising known amounts of the antigen. For such an assay method, for example, nephrometry, the competitive method, the immunometric method, the sandwich method, etc. are suitably used and in terms of sensitivity and specificity, it is preferred to use the sandwich method and the competitive method described later, particularly the sandwich method.

In the immobilization of antigens or antibodies, physical adsorption may be used. Alternatively, chemical binding that is conventionally used for immobilization/stabilization of proteins, enzymes, etc. may be used as well. Examples of the immobilizing carrier include insoluble polysaccharides such as agarose, dextran, cellulose, etc.; synthetic resins such as polystyrene, polyacrylamide, silicone, etc.; or glass; and the like.

In the sandwich method, the immobilized antibody of the present invention is reacted with a sample fluid (primary reaction), then with a labeled form of another antibody of the present invention (secondary reaction), and the amount or activity of the label on the immobilizing carrier is measured, whereby the amount of the NGF2/NT-3 in the sample fluid can be quantified. The order of the primary and secondary reactions may be reversed, and the reactions may be performed simultaneously or at staggered times. The methods of labeling and immobilization can be performed by the methods described above. In the immunoassay by the sandwich method, the antibody used for immobilized or labeled antibodies is not necessarily one species, but a mixture of two or more species of antibody may be used to increase the measurement sensitivity.

The antibody of the present invention can also be used in measuring system other than the sandwich method such as in the competitive method, the immunometric method, nephrometry, etc.

In the competitive assay, the NGF2/NT-3 in a sample fluid and a labeled form of the NGF2/NT-3 are reacted competitively against an antibody, an unreacted labeled antigen (F) is separated from an antibody-bound labeled antigen (B) (B/F separation), and the labeled amount of B or F is determined, thereby to quantify the NGF2/NT-3 in the sample fluid. The present reaction method includes a liquid phase method in which the B/F separation is carried out by using a soluble antibody as an antibody and using a secondary antibody against polyethylene glycol or against the antibody (primary antibody); and a solid phase method in which a solid-phased antibody is used as a primary antibody (direct method) or a soluble antibody is used as a primary antibody and a solid-phased antibody is used as a secondary antibody (indirect method).

In the immunometric assay, the NGF2/NT-3 in a sample fluid and a solid phase NGF2/NT-3 are competitively reacted with a given amount of a labeled form of the antibody followed by separating the solid phase from the liquid phase; or an NGF2/NT-3 in a sample fluid and an excess amount of labeled form of the antibody of the present invention are reacted, then a solid phase NGF2/NT-3 is added to bind an unreacted labeled form of the antibody of the present invention to the solid phase and the solid phase is then separated from the liquid phase. Thereafter, the labeled amount in any of the phases is measured to determine the protein of the present invention level in the sample fluid.

In the nephrometry, the amount of insoluble sediment, which is produced as a result of the antigen-antibody reaction in a gel or in a solution, is measured. Even when the amount of an NGF2/NT-3 in a sample fluid is small and only a small amount of the sediment is obtained, a laser nephrometry utilizing laser scattering can be suitably used.

For applying each of these immunological methods to the quantification method of the present invention, any particular conditions or procedures are not required. The measurement system of NGF2/NT-3 can be constructed by combining general conditions and procedures of each method with ordinary technical consideration of those of ordinary skill in the art. For the details of these general technical means, reference can be made to the following reviews and texts.

For example, Hiroshi Irie, ed. “Radioimmunoassay” (Kodansha, published in 1974), Hiroshi Irie, ed. “Sequel to the Radioimmunoassay” (Kodansha, published in 1979), Eiji Ishikawa, et al. ed. “Enzyme immunoassay” (Igakushoin, published in 1978), Eiji Ishikawa, et al. ed. “Immunoenzyme assay” (2nd ed.) (Igakushoin, published in 1982), Eiji Ishikawa, et al. ed. “Immunoenzyme assay” (3rd ed.) (Igakushoin, published in 1987), Methods in ENZYMOLOGY, Vol. 70 (Immunochemical Techniques (Part A)), ibid., Vol. 73 (Immunochemical Techniques (Part B)), ibid., Vol. 74 (Immunochemical Techniques (Part C)), ibid., Vol. 84 (Immunochemical Techniques (Part D: Selected Immunoassays)), ibid., Vol. 92 (Immunochemical Techniques (Part E: Monoclonal Antibodies and General Immunoassay Methods)), ibid., Vol. 121 (Immunochemical Techniques (Part I: Hybridoma Technology and Monoclonal Antibodies))(all published by Academic Press Publishing), etc. can be mentioned.

By using the antibody of the present invention as described above, it is possible to highly sensitively quantify the amount of NGF2/NT-3 in a testsolution, and hence the expression level of NGF2/NT-3 in the body of a test mammal. Therefore, if an immunoassay detects an increase in NGF2/NT-3 in the testsolution, the subject animal can be diagnosed as suffering from kidney cancer or urinary bladder cancer, or being likely to suffer from the same in the future.

The antibody of the present invention can also be used to diagnose other cancers wherein NGF2/NT-3 is overexpressed because it specifically recognizes NGF2/NT-3. Accordingly, the present invention also provides a diagnostic reagent for cancers that produce NGF2/NT-3, comprising the antibody of the present invention. In addition to kidney cancer and urinary bladder cancer, examples of cancers that produce NGF2/NT-3 include pancreatic cancer, prostate cancer, lung cancer, ovarian cancer, melanoma and the like.

The antibody of the present invention may be labeled with an appropriate labeling agent and administered to a subject mammal, and the localization of the antibody in the body can be examined by directly detecting (imaging) the labeling agent. As the labeling agent, a radioisotope having an appropriate half-life, for example, is preferably used. More preferably, the radioisotope is a nuclide in common use in various tomographies such as scintigraphy, single photon emission computed tomography (SPECT) and positron emission tomography (PET). Examples of nuclides for scintigraphy or SPECT include ^{99m}Tc, ²⁰¹Ti, ⁶⁷Ga, ¹¹¹In, ¹²³I, ¹³¹I, ¹²⁵I, ¹⁶⁹Yb, ¹⁸⁶Re, ⁹⁹Mo and the like, with preference given to ^{99mTc}. Examples of nuclides for PET include ¹⁵O, ¹³N, ¹¹C, ¹⁸F and the like.

Labeling of the antibody with a radioisotope can be achieved using a method known per se for each radioisotope. For example, the antibody can be labeled with ^{99m}Tc by, for example, the technique described in RADIOISOTOPES, 53: 155-178 (2004). Specifically, the antibody is coupled with an appropriate ligand (e.g., DTPA, HMPAO, DMSA, MAA and the like), via a linker as required, and this is enclosed in a container such as a vial. The pertechnetic acid ion (^{99m}TcO₄⁻) eluted from a ⁹⁹Mo—^{99m}Tc generator may be reduced to an oxidation number of +1, +3, +4 or +5 valence using an appropriate reducing agent (e.g., stannous chloride and the like), and injected and shaken in a container wherein a tracer compound is enclosed, whereby the ^{99m}Tc-labeled tracer compound can be obtained. ^{99m}Tc may be reacted directly with a desired ligand, or may be first reacted with a ligand with a weak potential for coordination, such as gluconic acid or tartaric acid, to produce a complex with the ligand, after which the complex may be reacted with a ligand with a strong potential for coordination to achieve ligand exchange. A linker in common use for the production of a technetium complex can be chosen and used as appropriate.

In addition to diagnostic imaging using a radioisotope, such as SPECT or PET, the antibody of the present invention can also be prepared as a non-radioactive contrast medium for diagnostic imaging such as MRI or CT, by being labeled with, for example, gadolinium, iodine, fluorine or the like. Alternatively, the antibody of the present invention can be labeled with a fluorescent substance such as green fluorescent protein (GFP) or a reporter that gives rise to a chemoluminescent substance (e.g., luciferase and the like).

The labeled antibody of the present invention can be prepared as a pharmaceutical composition the same manner as with the above-described antibody of the present invention, and can be administered via the same route of administration. Regarding the dose of the antibody in the form of, for example, an injectable preparation, intravenous administration at 0.001 to 1 mg/kg, preferably 0.005 to 0.2 mg/kg, per dose is favorable. When labeled with a radioisotope, the antibody exhibits a radioactivity intensity of about 0.0001 to 10 mCi, preferably about 0.01 to 0.1 mCi, per unit dose. The volume of the injectable formulation in a unit dosage is, for example, about 0.01 to 10 ml.

When a radioisotope is used as the labeling agent, antibody localization in the body of the subject animal is detected and imaged by, for example, scintigraphy, single photon emission computed tomography (SPECT), positron emission tomography (PET) or the like, preferably SPECT or PET. In the case of scintigraphy, administration of the labeled antibody is followed by delineation of the disposition thereof using a scintillation camera. In the case of SPECT and PET, transverse cross-sections are delineated using a respective dedicated tomograph. Timing of the start of imaging varies depending on the nuclide as the labeling agent, and is, for example, just after to 72 hours, preferably 5 minutes to 24 hours, more preferably 10 minutes to 4 hours after administration of the tracer.

When luciferase is used as the labeling agent, administration of the labeled antibody is followed by administration of luciferin, and the resulting chemoluminescence is visualized as digital images using a real time in vivo imaging apparatus (e.g., IVIS200 of Summit Pharmaceuticals International Corporation, and the like) equipped with an ultrasensitive cooling CCD camera, whereby the antibody can be detected. Also when another fluorescent or a luminescent substance is used as the labeling agent, the disposition of the antibody of the present invention can be delineated by detecting the label using a method known per se.

As a result, if accumulation of the antibody of the present invention is observed in the kidney or urinary bladder, the subject mammal can be diagnosed as suffering from kidney cancer or urinary bladder cancer, or being likely to suffer from the same in the future. If accumulation of the antibody of the present invention is observed in the vicinity of an organ or tissue, other than the kidney and urinary bladder, in which a cancer that produces NGF2/NT-3 can develop (e.g., pancreas, prostate, lung, ovary and the like), the subject mammal can be diagnosed as suffering from a cancer in the organ or tissue, or being likely to suffer from the same in the future.

In the description, where bases, amino acids, etc. are denoted by their codes, they are based on conventional codes in accordance with the IUPAC-IUB Commission on Biochemical Nomenclature or by the common codes in the art, examples of which are shown below. For amino acids that may have the optical isomer, L form is presented unless otherwise indicated.

DNA: deoxyribonucleic acid

cDNA: complementary deoxyribonucleic acid

A: adenine

T: thymine

G: guanine

C: cytosine

RNA: ribonucleic acid

mRNA: messenger ribonucleic acid

dATP: deoxyadenosine triphosphate

dTTP: deoxythymidine triphosphate

dGTP: deoxyguanosine triphosphate

dCTP: deoxycytidine triphosphate

ATP: adenosine triphosphate

EDTA: ethylenediaminetetraacetic acid

SDS: sodium dodecyl sulfate

Gly: glycine

Ala: alanine

Val: valine

Leu: leucine

Ile: isoleucine

Ser: serine

Thr: threonine

Cys: cysteine

Met: methionine

Glu: glutamic acid

Asp: aspartic acid

Lys: lysine

Arg: arginine

His: histidine

Phe: phenylalanine

Tyr: tyrosine

Trp: tryptophan

Pro: proline

Asn: asparagine

Gln: glutamine

pGlu: pyroglutamic acid

Sec: selenocysteine

The sequence identification numbers in the sequence listing of the description indicate the following sequences.

### [SEQ ID NO: 1]

This shows the base sequence of cDNA encoding human NGF2/NT-3.

### [SEQ ID NO: 2]

This shows the amino acid sequence of human NGF2/NT-3.

### [SEQ ID NO: 3]

This shows the base sequence of cDNA encoding the heavy chain of 3W3 antibody.

### [SEQ ID NO: 4]

This shows the amino acid sequence of the heavy chain of 3W3 antibody.

### [SEQ ID NO: 5]

This shows the base sequence of cDNA encoding the light chain of 3W3 antibody.

### [SEQ ID NO: 6]

This shows the amino acid sequence of the light chain of 3W3 antibody.

The present invention is explained in more detail in the following by referring to Examples, which are not to be construed as limitative.

### Examples

### Reference Example 1

### NGF2/NT-3 Neutralizing Activity of 3W3 Antibody

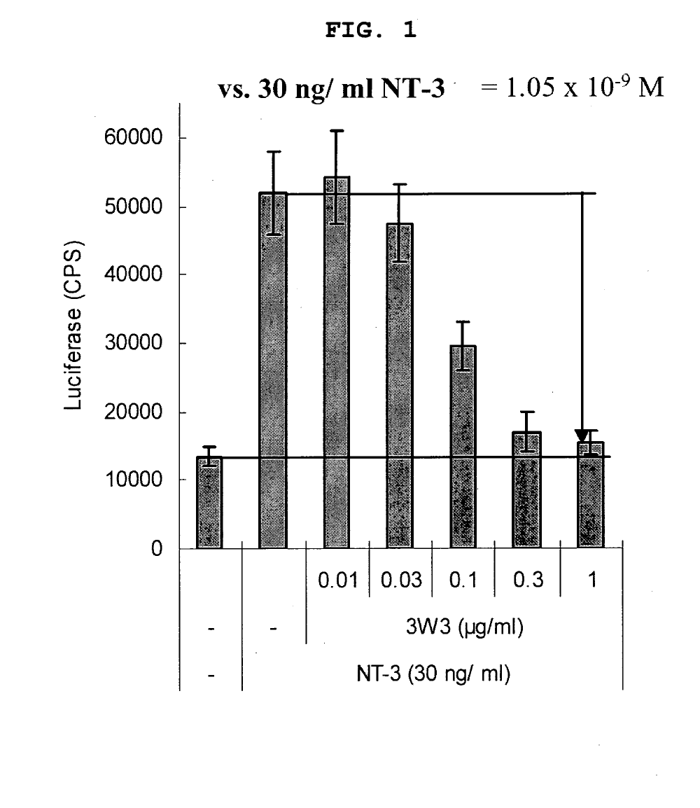

293T cells (2.4×10⁶ cells) were sown to a 10 cm dish and incubated in a DMEM comprising 10% FBS at 37° C. for 24 hours. The cells were transfected with three kinds of plasmid DNAs [12 μg of pCMV6-TrkC ty1 (Origene), 4 μg of pFA2-Elk1 (Stratagene), 8 μg of pFR-Luc (Stratagene)] using Lipofectamine 2000 (Invitrogen), after which they were further incubated at 37° C. for 65 hours. After being twice washed with Dulbecco's phosphate buffered saline (dPBS), the cells were dissociated using 1.5 ml of an enzyme-free cell-dissociation buffer (Invitrogen). After 9 ml of a DMEM comprising 0.5% FBS was added, the cells were recovered via centrifugation and re-suspended in 20 ml of a DMEM comprising 0.5% FBS. The cell suspension was dispensed to a 96-well plate (white bottom type) at 100 μl/well and incubated at 37° C. overnight. Mouse anti-human NGF2/NT-3 neutralizing antibody (3W3 antibody) was added to each well at various concentrations (0.01-1 μg/ml), recombinant human NGF2/NT-3 (30 ng/ml) was then added, and the cells were incubated at 37° C. for 24 hours, after which luciferase activity was determined using the Bright-Glo reagent (Promega). The results are shown in FIG. 1. The Elk1 signal activation by NGF2/NT-3 was inhibited dose-dependently by the 3W3 antibody, the IC50 being about 6.0×10⁻¹⁰ M.

### Reference Example 2

### Cloning and Sequencing of cDNAs of Heavy chain and light chain of 3W3 antibody

### (1) RNA Extraction and Purification

Total RNA was recovered from the hybridoma 3W3 in culture using RNAiso (TaKaRa), treated with DNase I, then with phenol and with chloroform, and purified by ethanol precipitation.

### (2) Cloning and Sequencing of Antibody Variable Region Fragment

An RT reaction was carried out by means of Reverse Transcriptase M-MLV (RNase H free) with 1 μg of the total RNA as the template, using a random primer (9-mer). The variable region was amplified by a PCR with a portion of this RT reaction liquid as the template, using LA Taq (TaKaRa). The primers for amplification of H chain and L chain used for the PCR reaction were in the primer set [Heavy Primers (Amersham 27-1586-01), Light Primer Mix (Amersham 27-1583-01)] attached to the Mouse scFv Module Recombinant Phage Antibody system (Amersham Biosciences K.K.).

Each amplified product obtained was subjected to agarose electrophoresis by a conventional method, after which it was purified from the gel, and subjected to TA cloning into the pMD20-T vector, and the sequence thereof was analyzed. The sequencing reaction was carried out using the Big Dye Terminator v3.1 Cycle Sequencing Kit (Applied Biosystems), and the data obtained were analyzed using the ABI PRISM3730 sequencer per the manufacturer's instructions in the attached protocol to determine the base sequences of the variable region fragments of the 3W3 antibody.

(3) Cloning and Sequencing of cDNA Ends by 5′-Race and 3′-RACE

Primers were prepared on the basis of the sequences of the H chain and L chain variable region fragments obtained in (2), and the gene sequences of both ends of the variable regions were analyzed by 5′-RACE and 3′-RACE.

The sequences (5′→3′) of the primers prepared are shown below.

Primer for H chain 5′-RACE:

| | |
|---|---|
| AGACGGCAGAGTCCACAGAGGTCAAAC | (SEQ ID NO: 7) |

Primer for H chain 3′-RACE:

| | |
|---|---|
| ACCTCTGTGGACTCTGCCGTCTATTTCTG | (SEQ ID NO: 8) |

Primer for L chain 5′-RACE:

| | |
|---|---|
| TGTCCCAGACCCACTGCCACTAAACC | (SEQ ID NO: 9) |

Primer for L chain 3′-RACE:

| | |
|---|---|
| CAACAGAAACCAGGACAGCCACCCA | (SEQ ID NO: 10) |

5′-RACE and 3′-RACE were performed using the SMART™ RACE cDNA Amplification Kit (Clontech) per the kit instructions in the attached protocol. The PCR products obtained were analyzed as described in (2) above.

(4) Cloning and Sequencing of Full-Length cDNA

Primers were prepared on the basis of the H chain and L chain 5′-side and 3′-side cDNA sequences obtained in (3), full-length cDNA was cloned by RT-PCR, and the sequence thereof was analyzed.

The sequences of the primers prepared are shown below.

| H chain 5′-primer: | |
|---|---|
| GATGATCAGTGTCCTCTCT | (SEQ ID NO: 11) |
| H chain 3′-primer: | |
| AGGGTCCCAAGGCAGTGCT | (SEQ ID NO: 12) |
| L chain 5′-primer: | |
| ATCCTCTCATCTAGTTCTC | (SEQ ID NO: 13) |
| L chain 3′-primer: | |
| GTGCAAAGACTCACTTTATTG | (SEQ ID NO: 14) |

An RT reaction was carried out by means of Reverse Transcriptase M-MLV (RNase H free) with 1 μg of the total RNA as the template, using a random primer (9-mer). The full length cDNA was amplified by a PCR with a portion of this RT reaction liquid as the template, using PrimeSTAR HS DNA polymerase (TaKaRa). The PCR products obtained were analyzed as described in (2) above.

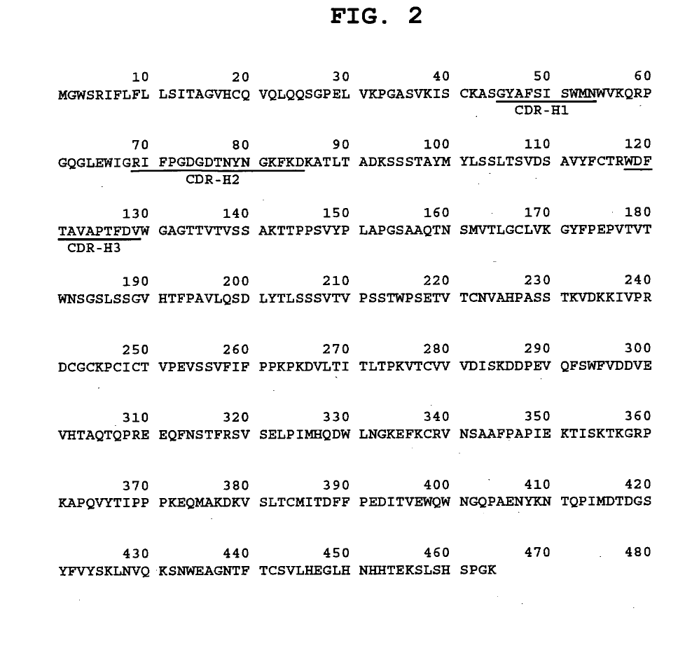

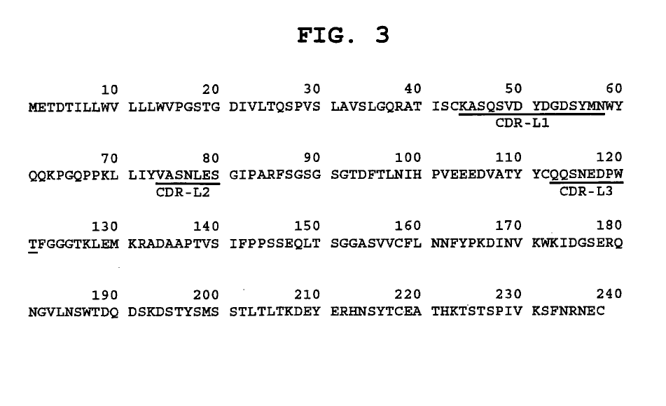

The base sequence and putative amino acid sequence of the heavy chain cDNA are shown by SEQ ID NO:3 and SEQ ID NO:4, respectively. The base sequence and putative amino acid sequence of the light chain cDNA are shown by SEQ ID NO:5 and SEQ ID NO:6, respectively. The complementarity determination region (CDR) of each of the heavy chain and light chain was predicted by a conventional method. The results are shown in FIG. 2 (heavy chain) and FIG. 3 (light chain).

### Example 1

### Antitumor Activity of Anti-Human NGF2/NT-3 Neutralizing Antibody Against Urinary Bladder Cancer Cells

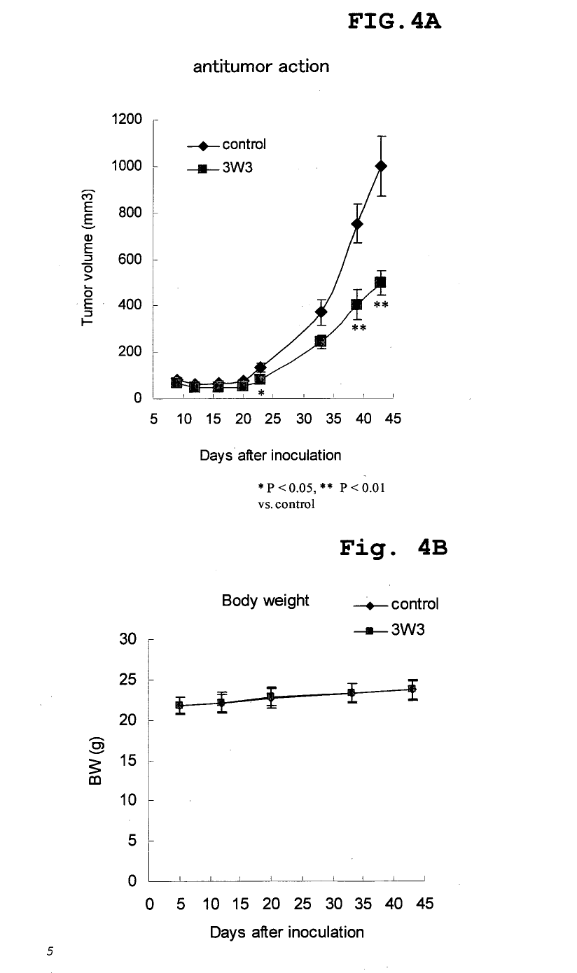

The antitumor action of an anti-human NGF2/NT-3 neutralizing antibody on TSU-Pr1 human urinary bladder cancer cells was examined using a nude mouse xenograft model. A suspension of TSU-Pr1 cells recovered after cultivation and proliferation in vitro was mixed with Matrigel (Invitrogen) in a ratio by volume of 1:1, and transplanted subcutaneously to female nude mice (5×10⁶ cells/mouse). On the following day, administration of the antibody was started. Using the mouse monoclonal antibody 3W3 as the anti-human NGF2/NT-3 neutralizing antibody, and the mouse anti-HIV-1 gp120 monoclonal antibody clone G115 (ATCC #CRL-2395) as the control antibody, twice-a-week intraperitoneal administration was performed at a dose of 400 μg/mouse. Starting on day 7 of transplantation, tumor size was measured intermittently using a vernie caliper, and tumor growth was evaluated in each of the groups respectively dosed with the above-described antibodies (tumor volume was calculated using the equation: tumor volume (mm³)=tumor length (mm)×[tumor width (mm)]²/2). As a result, in the 3W3 group, a significant suppression of tumor size was observed (FIG. 4A). Specifically, the tumor volume on day 42 of transplantation for the 3W3 group was 50% compared with the control group. During the study period, body weight gain was not suppressed (FIG. 4B), nor was there any unusual finding in the behavior or morphology. These results show that the anti-human NGF2/NT-3 neutralizing antibody is potentially a safe, effective therapeutic drug for urinary bladder cancer.

### Example 2

### Antitumor Activity of Anti-Human NGF2/NT-3 Neutralizing Antibody Against Kidney Cancer Cells

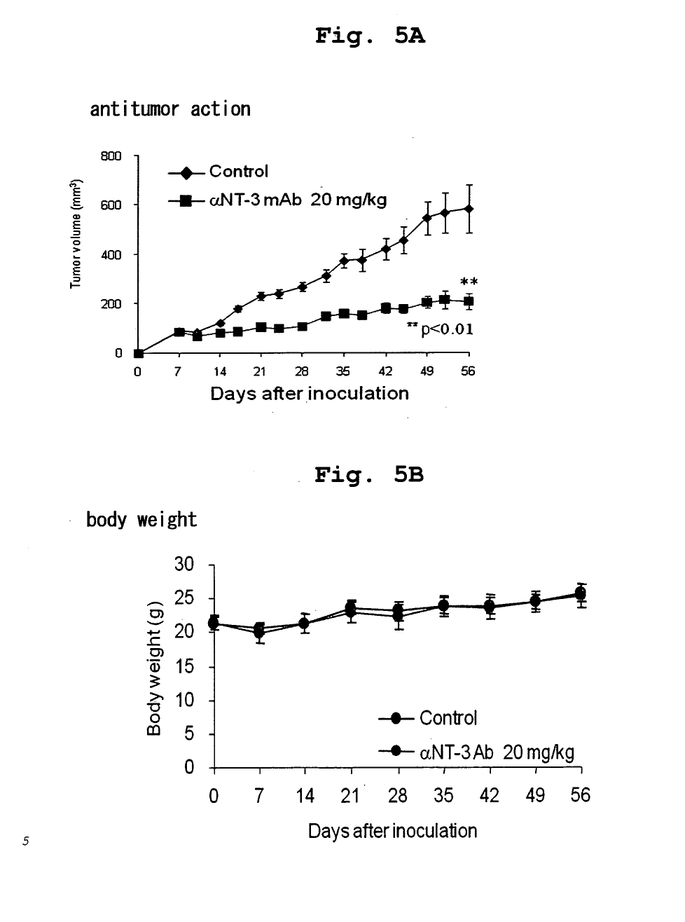

The antitumor action of an anti-human NGF2/NT-3 neutralizing antibody against ACHN human kidney cancer cells was examined using a nude mouse xenograft model. A suspension of ACHN cells recovered after cultivation and proliferation in vitro was transplanted subcutaneously to female nude mice (1×10⁷ cells/mouse). On the following day, administration of the antibody was started. Using the mouse monoclonal antibody 3W3 as the anti-human NGF2/NT-3 neutralizing antibody, and the mouse anti-HIV-1 gp120 monoclonal antibody clone G115 (ATCC #CRL-2395) as the control antibody, twice-a-week intravenous administration was performed at a dose of 20 mg/kg. Starting on day 7 of transplantation, tumor size was measured intermittently using a vernie caliper, and tumor growth was evaluated in each of the groups respectively dosed with the above-described antibodies (tumor volume was calculated using the equation: tumor volume (mm³)=tumor length (mm)×[tumor width (mm)]²/2). As a result, a significant suppression of tumor size was observed in the 3W3 group (FIG. 5A). Specifically, the tumor volume on day 56 of transplantation for the 3W3 group was 36% compared with the control group. During the study period, body weight gain was not suppressed (FIG. 5B), nor was there any unusual finding in the behavior or morphology. These results show that the anti-human NGF2/NT-3 neutralizing antibody is potentially a safe, effective therapeutic drug for kidney cancer.

### Reference Example 3

### Antitumor Activity of Anti-Human NGF2/NT-3 Neutralizing Antibody Against Small-Cell Lung Cancer Cells

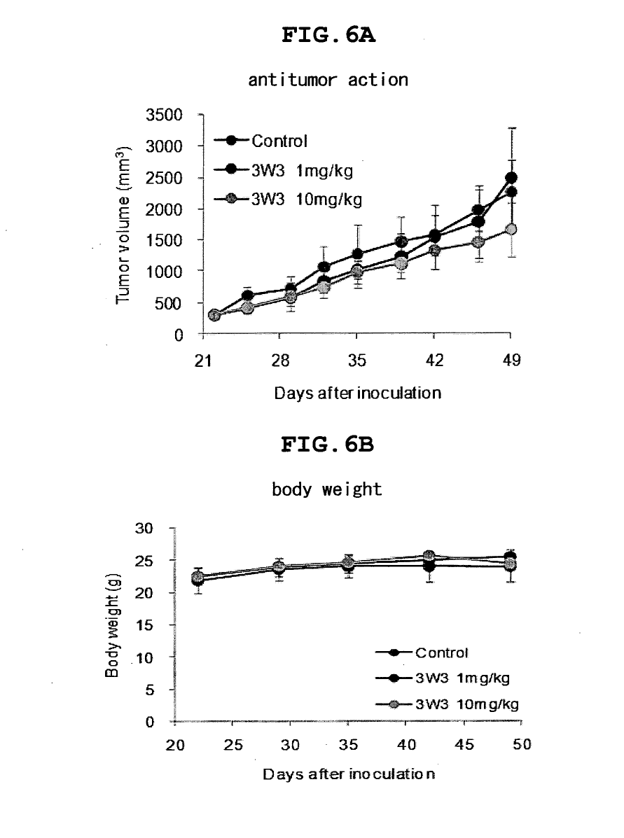

The antitumor action of an anti-human NGF2/NT-3 neutralizing antibody against COR-L88 human small-cell lung cancer cells was examined using a nude mouse xenograft model. A suspension of COR-L88 cells recovered after cultivation and proliferation in vitro was subcutaneously transplanted into the back of the neck of female nude mice (1×10⁷ cells/mouse). Tumor volume was measured 22 days after the subcutaneous transplantation, and the mice were radomized into 3 groups based on the tumor volume. Using the mouse monoclonal antibody 3W3 as the anti-human NGF2/NT-3 neutralizing antibody, twice-a-week intravenous administration was performed at a dose of 1 mg/kg or 10 mg/kg. Starting on grouping date, tumor size was measured intermittently using a vernie caliper, and tumor growth was evaluated in each of the groups respectively dosed with the above-described antibodies (tumor volume was calculated using the equation: tumor volume (mm³)=tumor length (mm)×[tumor width (mm)]²/2). As a result, a significant suppression of tumor size was observed in the 3W3 10 mg/kg administration group (FIG. 6A). Specifically, the tumor volume on day 49 of transplantation for the 3W3 group was 69% compared with the control group. During the study period, body weight gain was not suppressed (FIG. 6B), nor was there any unusual finding in the behavior or morphology. These results show that the anti-human NGF2/NT-3 neutralizing antibody is potentially a safe, effective therapeutic drug for small-cell lung cancer.

### INDUSTRIAL APPLICABILITY

The present invention provides an agent for the prophylaxis or treatment of kidney cancer or urinary bladder cancer, comprising an antibody against nerve growth factor 2/neurotrophin-3 or a partial peptide thereof or a salt thereof, which antibody neutralizes nerve growth factor 2/neurotrophin-3 and does not cross-react with the Nerve Growth Factor.

This application is based on U.S. provisional application No. 61/204,827 (filing date: Jan. 12, 2009), the contents of which are incorporated in full herein by this reference.

While the present invention has been described with emphasis on preferred embodiments, it is obvious to those skilled in the art that the preferred embodiments can be modified. The present invention intends that the present invention can be embodied by methods other than those described in detail in the present description. Accordingly, the present invention encompasses all modifications encompassed in the gist and scope of the appended “CLAIMS”.

The contents disclosed in any publication cited in the present description, including patents and patent applications, are hereby incorporated in their entireties by reference, to the extent that they have been disclosed herein.

1. An agent for the prophylaxis or treatment of kidney cancer, comprising an antibody against nerve growth factor 2/neurotrophin-3 or a partial peptide thereof or a salt thereof, which antibody neutralizes nerve growth factor 2/neurotrophin-3 and does not cross-react with the Nerve Growth Factor.

2. An agent for the prophylaxis or treatment of urinary bladder cancer, comprising an antibody against nerve growth factor 2/neurotrophin-3 or a partial peptide thereof or a salt thereof, which antibody neutralizes nerve growth factor 2/neurotrophin-3 and does not cross-react with the Nerve Growth Factor.

3. The agent of claim 1 or 2, wherein the antibody is a monoclonal antibody producible by the hybridoma 3W3 cell line (FERM BP-3932).

4. The agent of claim 1 or 2, wherein the nerve growth factor 2/neurotrophin-3 is human nerve growth factor 2/neurotrophin-3.

5. The agent of claim 1 or 2, wherein the antibody is a humanized antibody or a human antibody.

6. The agent of claim 1 or 2, wherein the antibody is a human/non-human chimeric antibody.

7. A diagnostic reagent of kidney cancer, comprising an antibody against nerve growth factor 2/neurotrophin-3 or a partial peptide thereof or a salt thereof, which antibody neutralizes nerve growth factor 2/neurotrophin-3 and does not cross-react with the Nerve Growth Factor.

8. A diagnostic reagent of urinary bladder cancer, comprising an antibody against nerve growth factor 2/neurotrophin-3 or a partial peptide thereof or a salt thereof, which antibody neutralizes nerve growth factor 2/neurotrophin-3 and does not cross-react with the Nerve Growth Factor.

9. A method for the prophylaxis or treatment of kidney cancer, comprising administering to a mammal an effective amount of an antibody against nerve growth factor 2/neurotrophin-3 or a partial peptide thereof or a salt thereof, which antibody neutralizes nerve growth factor 2/neurotrophin-3 and does not cross-react with the Nerve Growth Factor.

10. A method for the prophylaxis or treatment of urinary bladder cancer, comprising administering to a mammal an effective amount of an antibody against nerve growth factor 2/neurotrophin-3 or a partial peptide thereof or a salt thereof, which antibody neutralizes nerve growth factor 2/neurotrophin-3 and does not cross-react with the Nerve Growth Factor.

11. A method of diagnosing kidney cancer, comprising using an antibody against nerve growth factor 2/neurotrophin-3 or a partial peptide thereof or a salt thereof, which antibody neutralizes nerve growth factor 2/neurotrophin-3 and does not cross-react with the Nerve Growth Factor.

12. A method of diagnosing urinary bladder cancer, comprising using an antibody against nerve growth factor 2/neurotrophin-3 or a partial peptide thereof or a salt thereof, which antibody neutralizes nerve growth factor 2/neurotrophin-3 and does not cross-react with the Nerve Growth Factor.

13. A method of producing an agent for the prophylaxis or treatment of kidney cancer comprising isolating an antibody against nerve growth factor 2/neurotrophin-3 or a partial peptide thereof or a salt thereof, which antibody neutralizes nerve growth factor 2/neurotrophin-3 and does not cross-react with the Nerve Growth Factor.

14. A method of producing an agent for the prophylaxis or treatment of urinary bladder cancer comprising isolating an antibody against nerve growth factor 2/neurotrophin-3 or a partial peptide thereof or a salt thereof, which antibody neutralizes nerve growth factor 2/neurotrophin-3 and does not cross-react with the Nerve Growth Factor.

15. A method of producing a diagnostic reagent of kidney cancer comprising isolating an antibody against nerve growth factor 2/neurotrophin-3 or a partial peptide thereof or a salt thereof, which antibody neutralizes nerve growth factor 2/neurotrophin-3 and does not cross-react with the Nerve Growth Factor.

16. A method of producing a diagnostic reagent of urinary bladder cancer comprising isolating an antibody against nerve growth factor 2/neurotrophin-3 or a partial peptide thereof or a salt thereof, which antibody neutralizes nerve growth factor 2/neurotrophin-3 and does not cross-react with the Nerve Growth Factor.


SEQUENCE LISTING (16 sequences)

SEQ ID NO: 1 - DNA, 1349 bp, Homo sapiens
  CDS (212)..(1021)
  mat_peptide (665)..(1021)
agttgaagctcctctcccttccgaacagctccgcgcaccgccccgcgacgcagcccggcgcaactactttcttctctctcctttctttcttcctctcctttttcccctgctgggtagtggctgcggcggggtgggggagactttgaatgaccgagctcgcgtccacctttctcttcatgtcgacgtccctggaaacggccacacggatgccatggttacttttgccacgatcttacaggtgaacaaggtgatgtccatcttgttttatgtgatatttctcgcttatctccgtggcatccaaggtaacaacatggatcaaaggagtttgccagaagactcgctcaattccctcattattaagctgatccaggcagatattttgaaaaacaagctctccaagcagatggtggacgttaaggaaaattaccagagcaccctgcccaaagctgaggctccccgagagccggagcggggagggcccgccaagtcagcattccagccggtgattgcaatggacaccgaactgctgcgacaacagagacgctacaactcaccgcgggtcctgctgagcgacagcacccccttggagcccccgcccttgtatctcatggaggattacgtgggcagccccgtggtggcgaacagaacatcacggcggaaacggtacgcggagcataagagtcaccgaggggagtactcggtatgtgacagtgagagtctgtgggtgaccgacaagtcatcggccatcgacattcggggacaccaggtcacggtgctgggggagatcaaaacgggcaactctcccgtcaaacaatatttttatgaaacgcgatgtaaggaagccaggccggtcaaaaacggttgcaggggtattgatgataaacactggaactctcagtgcaaaacatcccaaacctacgtccgagcactgacttcagagaacaataaactcgtgggctggcggtggatacggatagacacgtcctgtgtgtgtgccttgtcgagaaaaatcggaagaacatgaattggcatctctccccatatataaattattactttaaattatatgatatgcatgtagcatataaatgtttatattgtttttatatattataagttgacctttatttattaaacttcagcaaccctacagtatataagcttttttctcaataaaatcagtgtgcttgccttccctcaggcctctcccatctgttaaaacttgttttgtgatccggctctcaggagtcactctgtaaaatctgtgtacaccagtattttgcattcagtattgtcaaggccatgactgttgttttagtaaacttgttaaaatcaaaaaaaaaaaaaaa

SEQ ID NO: 2 - protein, 270 aa, Homo sapiens
MVTFATILQVNKVMSILFYVIFLAYLRGIQGNNMDQRSLPEDSLNSLIIKLIQADILKNKLSKQMVDVKENYQSTLPKAEAPREPERGGPAKSAFQPVIAMDTELLRQQRRYNSPRVLLSDSTPLEPPPLYLMEDYVGSPVVANRTSRRKRYAEHKSHRGEYSVCDSESLWVTDKSSAIDIRGHQVTVLGEIKTGNSPVKQYFYETRCKEARPVKNGCRGIDDKHWNSQCKTSQTYVRALTSENNKLVGWRWIRIDTSCVCALSRKIGRT

SEQ ID NO: 3 - DNA, 1542 bp, Homo sapiens
  CDS (50)..(1441)
  mat_peptide (107)..(1441)
gatgatcagtgtcctctctacacagtccctgacgacactgactctaaccatgggatggagccggatctttctcttcctcctgtcaataactgcaggtgtccattgccaggtccagctgcagcagtctggacctgagctggtgaagcctggggcctcagtgaagatttcctgcaaagcttctggctacgcattcagtatctcttggatgaattgggtgaagcagaggcctggacagggtcttgagtggattggacggatttttcctggagatggagatactaattacaatggaaaattcaaggacaaggccacactgactgcagacaaatcctccagcacagcctacatgtatctcagcagtttgacctctgtggactctgccgtctatttctgcacaagatgggattttacggcagtagccccgaccttcgatgtctggggcgcagggaccacggtcaccgtctcctcagccaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagcacctggcccagcgagaccgtcacctgcaacgttgcccacccggccagcagcaccaaggtggacaagaaaattgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctggtaaatgatcccagtgtccttggagccctctggtcctacaggactctgacacctacctccacccctccctgtataaataaagcacccagcactgccttgggaccct

SEQ ID NO: 4 - protein, 464 aa, Homo sapiens
MGWSRIFLFLLSITAGVHCQVQLQQSGPELVKPGASVKISCKASGYAFSISWMNWVKQRPGQGLEWIGRIFPGDGDTNYNGKFKDKATLTADKSSSTAYMYLSSLTSVDSAVYFCTRWDFTAVAPTFDVWGAGTTVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 5 - DNA, 943 bp, Homo sapiens
  CDS (24)..(737)
  mat_peptide (84)..(737)
atcctctcatctagttctcagagatggagacagacacaatcctgctatgggtgctgctgctctgggttccaggctccactggtgacattgtgctgacccaatctccagtttctttggctgtgtctctaggtcagagggccaccatctcctgcaaggccagccaaagtgttgattatgatggtgatagttatatgaactggtaccaacagaaaccaggacagccacccaaactcctcatctatgttgcatccaatctagaatctgggatcccagccaggtttagtggcagtgggtctgggacagacttcaccctcaacatccatcctgtggaggaggaggatgttgcaacctattactgtcagcaaagtaatgaggatccgtggacgttcggtggaggcaccaagctggaaatgaaacgagctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgttagagacaaaggtcctgagacgccaccaccagctccccagctccatcctatcttcccttctaaggtcttggaggcttccccacaagcgacctaccactgttgcggtgctccaaacctcctccccacctccttctcctcctcctccctttccttggcttttatcatgctaatatttgcagaaaatattcaataaagtgagtcttgac

SEQ ID NO: 6 - protein, 238 aa, Homo sapiens
METDTILLWVLLLWVPGSTGDIVLTQSPVSLAVSLGQRATISCKASQSVDYDGDSYMNWYQQKPGQPPKLLIYVASNLESGIPARFSGSGSGTDFTLNIHPVEEEDVATYYCQQSNEDPWTFGGGTKLEMKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 7 - DNA, 27 bp, Artificial Sequence
  Description of Artificial Sequence Synthetic primer
agacggcagagtccacagaggtcaaac

SEQ ID NO: 8 - DNA, 29 bp, Artificial Sequence
  Description of Artificial Sequence Synthetic primer
acctctgtggactctgccgtctatttctg

SEQ ID NO: 9 - DNA, 26 bp, Artificial Sequence
  Description of Artificial Sequence Synthetic primer
tgtcccagacccactgccactaaacc

SEQ ID NO: 10 - DNA, 25 bp, Artificial Sequence
  Description of Artificial Sequence Synthetic primer
caacagaaaccaggacagccaccca

SEQ ID NO: 11 - DNA, 19 bp, Artificial Sequence
  Description of Artificial Sequence Synthetic primer
gatgatcagtgtcctctct

SEQ ID NO: 12 - DNA, 19 bp, Artificial Sequence
  Description of Artificial Sequence Synthetic primer
agggtcccaaggcagtgct

SEQ ID NO: 13 - DNA, 19 bp, Artificial Sequence
  Description of Artificial Sequence Synthetic primer
atcctctcatctagttctc

SEQ ID NO: 14 - DNA, 21 bp, Artificial Sequence
  Description of Artificial Sequence Synthetic primer
gtgcaaagactcactttattg

SEQ ID NO: 15 - protein, 55 aa, Artificial Sequence
  Description of Artificial Sequence Synthetic polypeptide
SGGGGGGGGGGSGGGGGGGGGGSGGGGGGGGGGSGGGGGGGGGGSGGGGGGGGGG

SEQ ID NO: 16 - protein, 55 aa, Artificial Sequence
  Description of Artificial Sequence Synthetic polypeptide
GGGGGGGGGGSGGGGGGGGGGSGGGGGGGGGGSGGGGGGGGGGSGGGGGGGGGGS